(19) United States
(12) Patent Application Publication
(10) Pub. No.: US 2022/0404084 A1
(43) Pub. Date: Dec. 22, 2022

(54) METHODS, SYSTEMS, DEVICES, AND FORMULATIONS FOR CRYOGENIC FLUIDS

(71) Applicant: Glacia, Inc., Snohomish, WA (US)

(72) Inventors: Craig Clinton Rominger, Snohomish, WA (US); Derek Pagenkopf, Bothell, WA (US)

(73) Assignee: Glacia, Inc., Snohomish, WA (US)

(21) Appl. No.: 17/866,241

(22) Filed: Jul. 15, 2022

Related U.S. Application Data
(60) Provisional application No. 63/209,243, filed on Jun. 10, 2021.

(51) Int. Cl. F25C 1/147; A61F 7/00; A61L 2/26; A61L 2/10; A01N 1/02

(52) U.S. Cl. CPC F25C 1/147; A61F 7/00; A61L 2/26; A61L 2/10; A01N 1/0284; F25C 2400/12; A61F 2007/0063; A61L 2202/11; A61L 2202/21

(57) ABSTRACT

A cryogenic fluid composition may include water (H20), and at least one salt. The ratio of water to the at least one salt is approximately between 1% and 6% salt with the remainder water. A cryogenic fluid production device may include a cylindrical housing, and a heat exchanger disposed within the cylindrical housing. The heat exchanger may include an inlet, a channel, and an outlet. A coolant may be conveyed through the inlet, the channel, and the outlet of the heat exchanger. The cryogenic fluid production device may further include an interior wall, and an auger disposed within the interior wall of the heat exchanger.

### RELATED APPLICATIONS

This application claims priority to and incorporates U.S. Provisional Patent Application 63/209,243, filed Jun. 10, 2021, entitled “Methods, Systems, Devices, and Formulations for Cryogenic Fluids,” in its entirety by reference.

### TECHNICAL FIELD

The present disclosure relates generally to therapeutic devices, systems, processes, and formulations. Specifically, the present disclosure relates to systems, methods and formulations creating a therapeutic, homogeneous, cryogenic fluid (e.g., cryofluid).

### BACKGROUND

Cryotherapy may include the use of cryogenic fluid such as water (e.g., ice) and other non-toxic refrigerants to treat a variety of tissue lesions. Cryotherapy may be used in an effort to relieve muscle pain, sprains, and swelling after soft tissue damage or surgery. For example, cryotherapy may be used to accelerate recovery in athletes post exercise. Cryotherapy decreases the temperature of tissue surface to minimize hypoxic cell death, edema accumulation, and muscle spasms, all of which ultimately alleviate discomfort and inflammation. Some cryogenic systems may be used to freeze the cryogenic fluid.

Augers may include any rotating, helical screw. The auger may be housed in a cylindrical housing of a cryogenic system to move material through the cylindrical housing. In some examples, the auger may also be used to remove material from the inside of the cylindrical housing. The rotation of the auger within the cylindrical housing causes the material to be pulled from the sides of the cylindrical housing and from a first end of the cylindrical housing to a second end of the cylindrical housing. In some examples, the auger and cylindrical housing may be used to create pressure in the material being moved through the cylindrical housing by forcing the material to the second end of the cylindrical housing.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates a cryogenic fluid creation system, according to an example of the principles described herein.

FIG. 2 illustrates a mobile cryogenic fluid creation system, according to an example of the principles described herein.

FIG. 3 illustrates a mobile cryogenic fluid creation system, according to an example of the principles described herein.

FIG. 4 illustrates a cryogenic fluid creation system, according to an example of the principles described herein.

FIG. 5 illustrates the cryogenic fluid creation system of FIG. 4 with a number of guards removed to expose internal elements of the cryogenic fluid creation system, according to an example of the principles described herein.

FIG. 6 illustrates a cryogenic fluid creation system, according to an example of the principles described herein.

FIG. 7 illustrates a cryogenic fluid creation system, according to an example of the principles described herein.

FIG. 8 illustrates an auger for use in a cryogenic fluid creation system, according to an example of the principles described herein.

FIG. 9 illustrates a number of different views of the auger of FIG. 8 including a cross-sectional side view, an end-on view, and a side view including threads depicted in solid and in ghost, according to an example of the principles described herein.

FIG. 10 illustrates an entry shaft coupled to a top of the auger of FIGS. 8 and 9, according to an example of the principles described herein.

FIG. 11 illustrates an exit shaft coupled to a bottom of the auger of FIGS. 8 and 9, according to an example of the principles described herein.

FIG. 12 illustrates a cryogenic fluid generator assembly of a cryogenic fluid creation system, according to an example of the principles described herein.

FIG. 13 illustrates a heat exchanger for use in a cryogenic fluid creation system, according to an example of the principles described herein.

FIG. 14 illustrates a shell of a heat exchanger for use in a cryogenic fluid creation system, according to an example of the principles described herein.

FIG. 15 illustrates a core of a heat exchanger for use in a cryogenic fluid creation system, according to an example of the principles described herein.

FIG. 16 illustrates a base of a heat exchanger for use in a cryogenic fluid creation system, according to an example of the principles described herein.

FIG. 17 illustrates a bottom flange of a heat exchanger for use in a cryogenic fluid creation system, according to an example of the principles described herein.

FIG. 18 illustrates a top flange of a heat exchanger for use in a cryogenic fluid creation system, according to an example of the principles described herein.

FIG. 19 illustrates a flange half coupling including national pipe tapered (NPT) threads for coupling pressurized fluid lines within a cryogenic fluid creation system, according to an example of the principles described herein.

FIG. 20 illustrates a fluid filter assembly of a cryogenic fluid creation system, according to an example of the principles described herein.

FIG. 21 illustrates a frame assembly of a cryogenic fluid creation system, according to an example of the principles described herein.

FIG. 22 illustrates a check valve and drain valve assembly of a cryogenic fluid creation system, according to an example of the principles described herein.

FIG. 23 illustrates a resistance temperature detector (RTD) assembly of a cryogenic fluid creation system, according to an example of the principles described herein.

FIG. 24 illustrates a reservoir assembly of a cryogenic fluid creation system, according to an example of the principles described herein.

FIG. 25 illustrates a pump assembly of a cryogenic fluid creation system, according to an example of the principles described herein.

FIG. 26 illustrates an ultraviolet germicidal irradiation (UVGI) assembly of a cryogenic fluid creation system, according to an example of the principles described herein.

FIGS. 27 and 28 illustrates a water filter housing of a cryogenic fluid creation system, according to an example of the principles described herein.

FIG. 29 illustrates electrical box bracket of a cryogenic fluid creation system, according to an example of the principles described herein.

FIG. 30 illustrates a valve bracket of a cryogenic fluid creation system, according to an example of the principles described herein.

FIG. 31 illustrates an electrical box mount of a cryogenic fluid creation system, according to an example of the principles described herein.

FIG. 32 illustrates a reservoir shelf and sanitizer mount of a cryogenic fluid creation system, according to an example of the principles described herein.

FIG. 33 illustrates a base plate of a cryogenic fluid creation system, according to an example of the principles described herein.

FIG. 34 illustrates a front, lower left housing guard of a cryogenic fluid creation system, according to an example of the principles described herein.

FIG. 35 illustrates a front, upper housing guard of a cryogenic fluid creation system, according to an example of the principles described herein.

FIG. 36 illustrates a front, upper housing guard of a cryogenic fluid creation system, according to an example of the principles described herein.

FIG. 37 illustrates a front, lower right housing guard of a cryogenic fluid creation system, according to an example of the principles described herein.

FIG. 38 illustrates a right side housing guard of a cryogenic fluid creation system, according to an example of the principles described herein.

FIG. 39 illustrates a left side housing guard of a cryogenic fluid creation system, according to an example of the principles described herein.

FIG. 40 illustrates a rear housing guard of a cryogenic fluid creation system, according to an example of the principles described herein.

FIG. 41 illustrates a top housing guard of a cryogenic fluid creation system, according to an example of the principles described herein.

FIG. 42 illustrates a heat exchanger system of a cryogenic fluid creation system, according to an example of the principles described herein.

FIG. 43 illustrates a heat exchanger of a cryogenic fluid creation system, according to an example of the principles described herein.

FIG. 44 illustrates an internal chamber of a heat exchanger of a cryogenic fluid creation system, according to an example of the principles described herein.

FIG. 45 illustrates a number of intermediate sleeves of an internal chamber of a heat exchanger of a cryogenic fluid creation system, according to an example of the principles described herein.

### DESCRIPTION OF EXAMPLE EMBODIMENTS

Examples described herein provide for systems and methods related to a simplified cryogenic system for cooling and freezing cryogenic fluid where the cryogenic fluid, once frozen, is scraped from an interior wall of a cylindrical housing by an auger housed and mechanically rotated within the cryogenic system. The cryogenic system may include a heat exchange unit contained in an outer housing and in thermal coupling with the cylindrical housing and/or the auger. The present systems and methods further provide for a sealed, rapid heat exchange system including modular, self-aligning auger including a number of indexable end mills. Still further, the present systems and methods provide an angled push unit for expulsion of frozen material from the cylindrical housing of the cryogenic system. Further, the present systems and methods provide a number of chilling coils surrounding the auger and/or the cylindrical housing to freeze the cryogenic fluid in order to produce the therapeutic, frozen cryogenic fluid.

### Overview

In the examples described herein, a cryogenic fluid production device or system may be used to produce a cryogenic fluid or slurry from a cryogenic fluid composition. The cryogenic fluid composition may include, for example, water, filtered water, sanitized water, at least one salt, at least one alcohol, at least on sugar, at least one therapeutic, and combinations thereof. The cryogenic fluid or slurry may include nano-sized particles that may enter tissues and organs for therapeutic purposes.

Examples described herein provide a cryogenic fluid production device including a cylindrical housing and a heat exchanger disposed within the cylindrical housing. The heat exchanger may include an inlet, a channel, and an outlet. A coolant may be conveyed through the inlet, the channel, and the outlet of the heat exchanger. The cryogenic fluid production device may further include an interior wall, and an auger disposed within the interior wall of the heat exchanger.

The auger may include at least one helical ridge that interfaces with the ice particles gathered on the interior wall. The at least one helical ridge forces a cryogenic fluid composition introduced into an interior of the interior wall in a direction opposite a gravitational force. A distance between the helical ridge of the auger and the wall may be between 0.005 in. to 0.015 in. The interior wall may be textured.

The cryogenic fluid production device may further include a processor, and a non-transitory computer-readable media storing instructions that, when executed by the processor, causes the processor to perform operations. The operations may include displaying, via a user interface, information defining a formulation of a cryogenic fluid introduced into the cryogenic fluid device, a rotational speed of the auger, a status of a cryogenic fluid mixing process, a status of a cryogenic fluid cooling process, and combinations thereof. The cryogenic fluid device may be ambulatory.

The cryogenic fluid production device may further include an ultraviolet germicidal irradiation (UVGI) assembly to sterilize at least one component of a cryogenic fluid composition. The cryogenic fluid production device may further include at least one filter to filter at least one component of a cryogenic fluid composition.

Examples described herein also provide a therapeutic method. The therapeutic method may include applying a cryogenic fluid to an organ tissue. The cryogenic fluid is formed between 20° F. and 31° F. Further, the cryogenic fluid may include at least one of water (H₂0) and at least one salt. The ratio of water to the at least one salt may be approximately between 1% and 6% salt with the remainder water. The method may further include applying the cryogenic fluid directly to a tissue of an organ, indirectly to the organ tissue, and combinations thereof. At least one of a temperature of the cryogenic fluid composition, a density of the cryogenic fluid composition, a viscosity of the cryogenic fluid composition, a size of solid particles within the cryogenic fluid composition or combinations thereof may be effected by adjusting at least one of a temperature of the cryogenic fluid composition as introduced into a cryogenic fluid composition device, a rotational speed of an auger within the cryogenic fluid composition device, a temperature of a heat exchange element of the cryogenic fluid composition device, or combinations thereof.

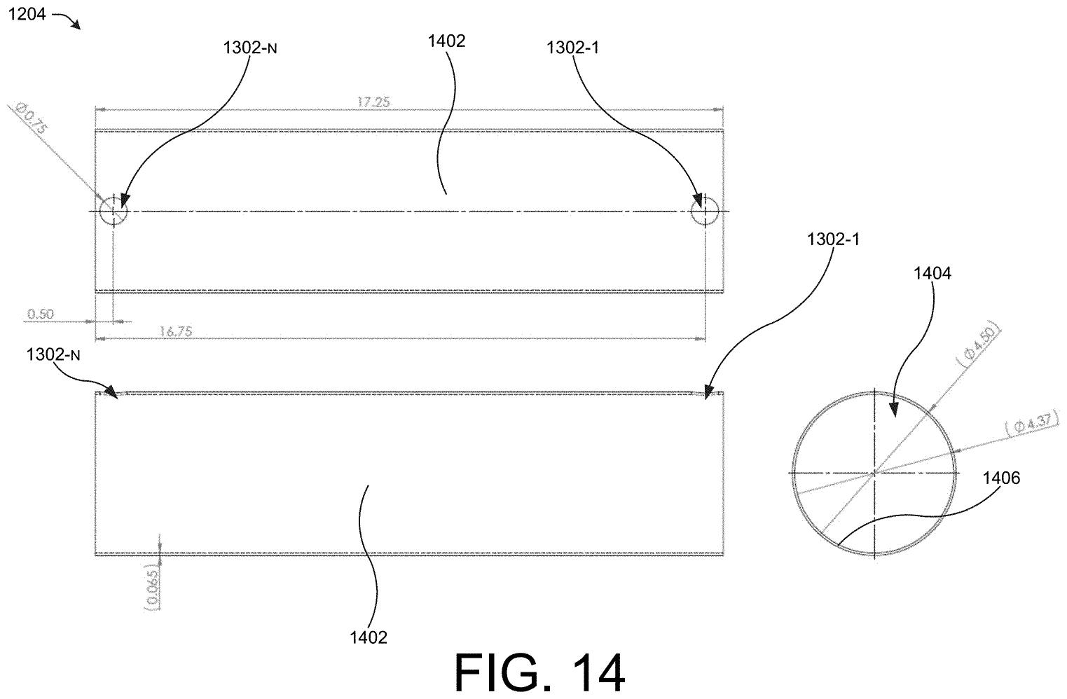

Examples described herein also provide a cryogenic fluid composition. The cryogenic fluid composition may include water (H₂0), and at least one salt. The ratio of water to the at least one salt is approximately between 1% and 6% salt with the remainder water. The ratio may be measured by weight. The ratio may be measured by volume. The cryogenic fluid may be formed between 20° F. and 31° F. The shape of ice particles within the cryogenic fluid may include at least one of approximately round, oblong, or globular, and may include a roughness average (RA) of between 63 RA and 125 RA. The roughness (e.g., small scratches) of the interior wall 1406 of FIG. 14 at this range allows rapid nucleation and ice crystal formation. This, in turn, allows adhesion of the above-mentioned ice crystal structures and subsequent fracture of the ice crystal by the ice generating auger into molecular nanoparticles (e.g., nano-ice). The diameter of ice particles within the cryogenic fluid may be between 1 nanometer and 900 micrometers. The at least one salt may include Sodium Chloride (NaCl) and magnesium sulfate (MgSO₄). The cryogenic fluid may further include at least one of an alcohol, a sugar, the at least one salt, and combinations thereof.

The cryogenic fluid may further include at least one of methylsulfonylmethane (MSM), glucosamine, aloe including pure aloe, Epsom salts, trehalose, autologous cultured chondrocytes, cytokines for wound healing (e.g., derma gel, silvasorb, chlorhexidine 2%/4%, steroid creams), botulinum toxin type A, onabotulalinumtoxina (e.g., Botox), baclofen, tizanidine, cyclobenzaprine, iodine preparations (e.g., tincture of iodine, potassium iodide, iodophors), copper preparations (e.g., copper sulfate, copper naphthenate, cuprimyxin), sulfur preparations (e.g., monosulfiram, benzoyl disulfide), phenols (e.g., phenol, thymol), fatty acids and salts (e.g., propionates, undecylenates), organic acids (e.g., benzoic acid, salicylic acids), dyes (e.g., crystal [gentian] violet, carbolfuchsin), hydroxyquinolines (e.g., iodochlorhydroxyquin), nitrofurans (e.g., nitrofuroxine, nitrofurfurylmethyl ether), imidazoles (e.g., miconazole, tioconazole, clotrimazole, econazole, thiabendazole), polyene antibiotics (e.g., amphotericin B, nystatin, pimaricin, candicidin, hachimycin), allylamines (e.g., naftifine, terbinafine), thiocarbamates (e.g., tolnaftate), and miscellaneous agents (e.g., acrisorcin, haloprogin, ciclopirox, olamine, dichlorophen, hexetidine, chlorphenesin, triacetin, polynoxylin, amorolfine, Triclosan, Microban, Iodine, O-phenylphenol, Hydronium, Dakin's Solution, hydrogen peroxide, honey, vinegar, essential oils, Erythromycin (e.g., antibiotics), mesenchymal stem cells (e.g., MSCs), platelet-rich plasma (PRP), autologous conditioned serum (ACS) and autologous protein solution (APS), chlorhexidine, dermatophilus congolensis, and combinations thereof.

Methylsulfonylmethane (MSM) is an organosulfur compound with the formula (CH₃)₂SO₂. MSM is also known by several other names including methyl sulfone and dimethyl sulfone (DMSO2). This colorless solid features the sulfonyl functional group and is the simplest of the sulfones. It is considered relatively inert chemically and is able to resist decomposition at elevated temperatures. It occurs naturally in some primitive plants, is present in small amounts in many foods and beverages and is marketed as a dietary supplement. Small-scale studies of possible treatments with MSM have been conducted on both animals and humans. These studies of MSM have suggested some benefits, particularly for treatment of oxidative stress and osteoarthritis.

Additionally, the techniques described in this disclosure may be performed as a method and/or by a system having non-transitory computer-readable media storing computer-executable instructions that, when executed by one or more processors, performs the techniques described above.

### Example Embodiments

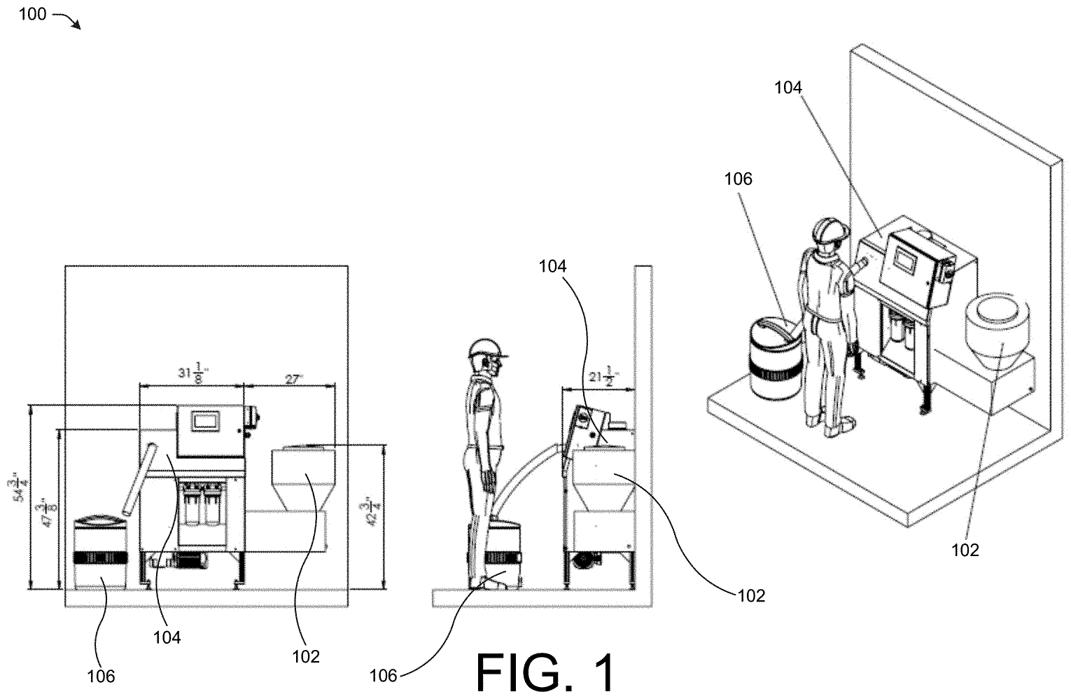

Turning now to the figures, FIG. 1 illustrates a cryogenic fluid creation system 100, according to an example of the principles described herein. The cryogenic fluid creation system 100 may take a number of forms including industrial-sized systems, in-office-seized systems, mobile systems, and other types of systems that provide for the creation of a cryogenic fluid such as those compositions described herein. The example of the cryogenic fluid creation system 100 of FIG. 1 may include a hopper 102. In one example, the hopper 102 may include any type of autonomous mix and feed system in which the cryogenic fluid compositions described herein may be mixed and fed into a main cryogenic fluid generator 104. The main cryogenic fluid generator 104 may include a number of elements described below that generate the cryogenic fluid formed from the cryogenic fluid compositions described herein. As used in the present specification and in the appended claims, the term “cryogenic fluid composition” is meant to be understood broadly as any composition before being introduced into the main cryogenic fluid generator 104. Further, as used in the present specification and in the appended claims, the term “cryogenic fluid” is meant to be understood broadly as any composition created by the main cryogenic fluid generator 104. The cryogenic fluid may include nano-sized frozen particles of the cryogenic fluid composition. In this state, the cryogenic fluid is generated and is able to flow out of the cryogenic fluid creation system 100 as a slurry or fluid.

Further, the cryogenic fluid creation system 100 may include a collection reservoir 106 for collecting the cryogenic fluid once it is dispensed from the main cryogenic fluid generator 104. With the cryogenic fluid, an individual may treat a number of musculoskeletal injuries such as, for example, injuries to muscles, bones, cartilage, ligaments, tendons and connective tissues of all kinds and severities, muscle strains, and muscle fatigue, among a myriad of other types of injuries. Further, the cryogenic fluid may be used to preserve organs for transplant. For example, prior to the organ being removed from the donor the organ may be flushed free of blood using the cryogenic fluid as an ice-cold preservation solution that contains electrolytes and/or nutrients. Further, after harvesting the organ, the organ may be placed in a sterile container along with additional cryogenic fluid and transported to a transplant center for implant into a recipient. Other uses and purposes for the cryogenic fluid are described herein.

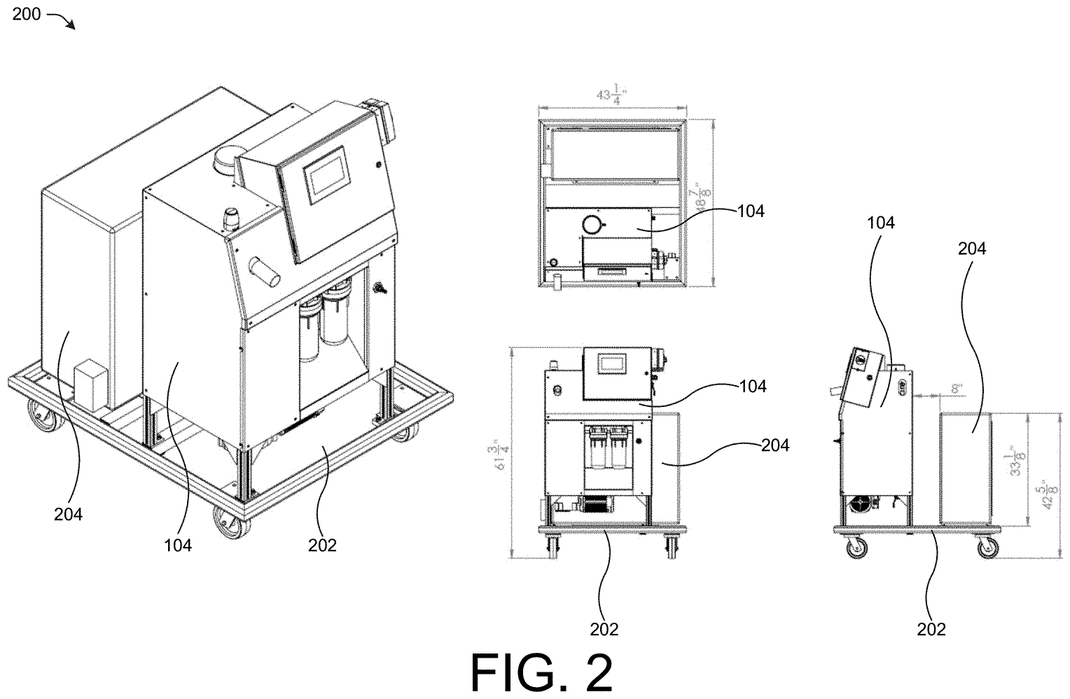

FIG. 2 illustrates a mobile cryogenic fluid creation system 200, according to an example of the principles described herein. The example of the mobile cryogenic fluid creation system 200 of FIG. 2 may include a main cryogenic fluid generator 104 seated or carried by a cart 202 or other conveying device. This allows the mobile cryogenic fluid creation system 200 to be moved from one location to another whether those two locations are within the same building or area, or very distant areas. This allows for the mobile cryogenic fluid creation system 200 to be brought to a location at which a patient or other user may require treatment rather than having to move the patient to a facility wherein the mobile cryogenic fluid creation system 200 is located. The mobile cryogenic fluid creation system 200, in one example, may further include a power generation device 204 that may power the main cryogenic fluid generator 104 in instances where mains power provided via a power outlet is available.

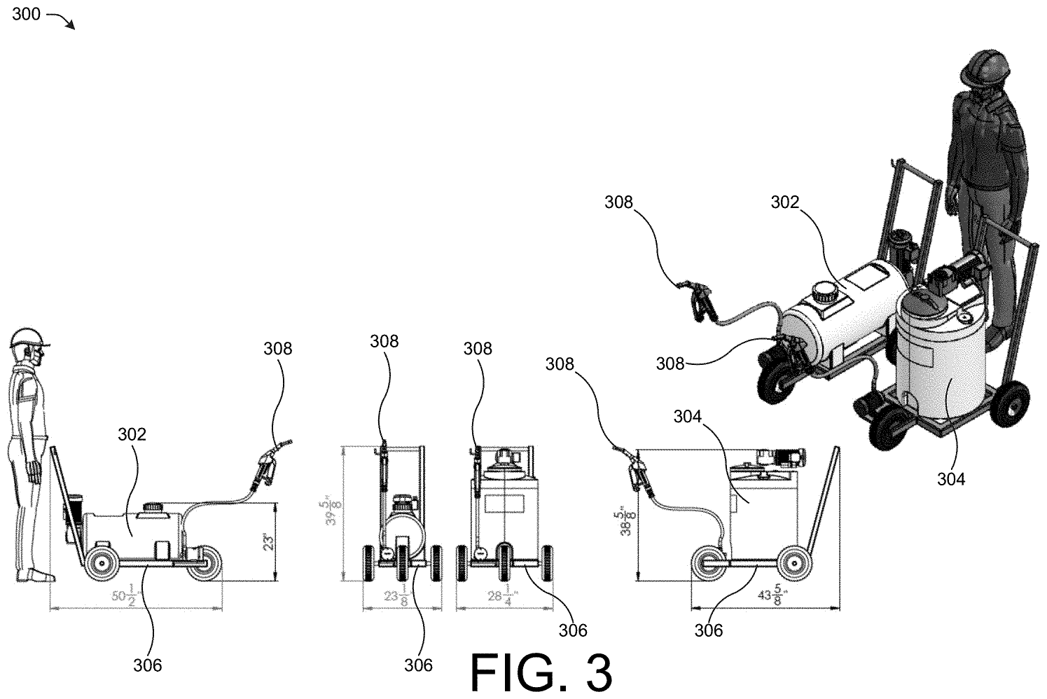

FIG. 3 illustrates a mobile cryogenic fluid creation system 300, according to an example of the principles described herein. The mobile cryogenic fluid creation system 300 may include a two differ canister-style devices including a horizontally-oriented canister device 302 and a vertically-oriented canister device 304. The horizontally-oriented canister device 302 and a vertically-oriented canister device 304 may each include a trolley 306 with varying dimensions that allow for the horizontally-oriented canister device 302 and a vertically-oriented canister device 304 to be conveyed in a manner similar to the example of FIG. 2. The mobile cryogenic fluid creation system 300 may further include dispensing devices 308 including hoses, pump-activating handles, pumps, and other devices that provide for the dispensing of the cryogenic fluid generated within the horizontally-oriented canister device 302 and a vertically-oriented canister device 304, respectively.

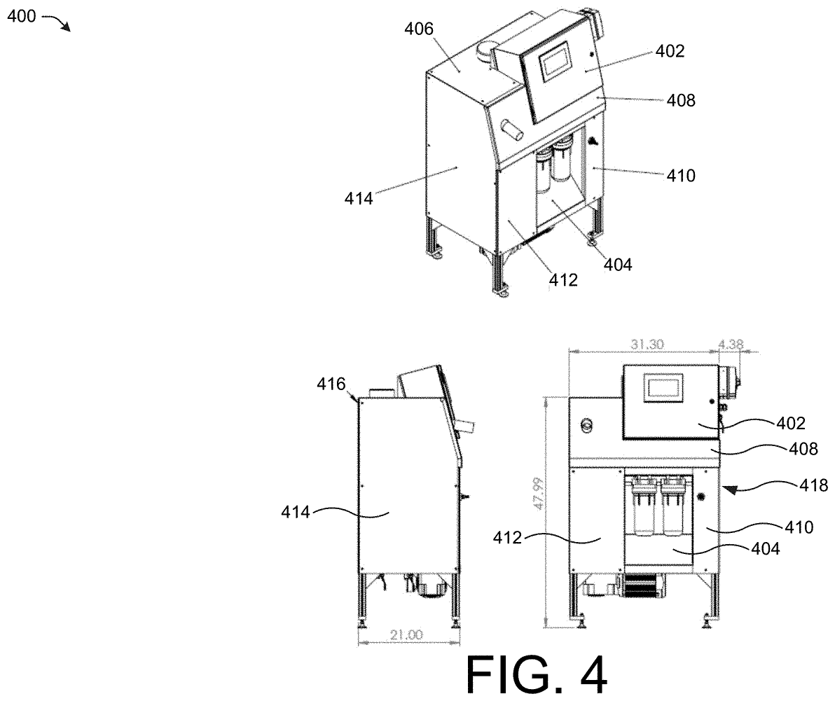

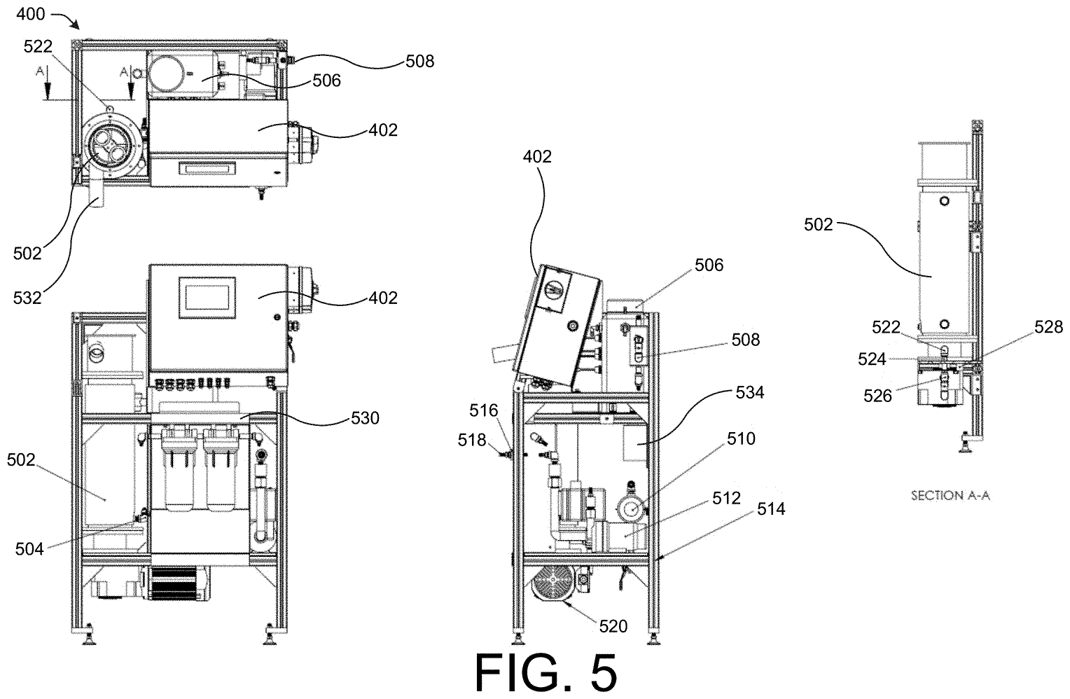

FIG. 4 illustrates a cryogenic fluid creation system 400, according to an example of the principles described herein. FIG. 5 illustrates the cryogenic fluid creation system of FIG. 4 with a number of guards removed to expose internal elements of the cryogenic fluid creation system 400, according to an example of the principles described herein. The cryogenic fluid creation system 400 of FIGS. 4 and 5 may be used to describe the various internal elements of the examples of the cryogenic fluid creation systems described herein. Similar elements of the cryogenic fluid creation system 400 of FIGS. 4 and 5 may be included in other examples described herein.

The cryogenic fluid creation system 400 may include an electrical control assembly 402 used to control the various elements of the cryogenic fluid creation system 400 described herein including all the elements used to prepare, convey, sanitize, pump, and generate the cryogenic fluid composition and/or the cryogenic fluid.

Fluid may be introduced into the cryogenic fluid creation system 400 via an adapter 518 coupled to the front lower right housing guard 410 via a bulkhead coupler 516. In one example, the adapter 518 may include a ⅜ inch (in.) NPT to barbed hose adapter that allows a hose to be selectively attached and removed from the cryogenic fluid creation system 400 via a number of barbs formed on the adapter 518 that acts as a gripper that holds the hose coupled to the adapter 518. In one example, the fluid may include water, water compositions, the cryogenic fluid composition, other chemical elements, and combinations thereof.

The water introduced into the cryogenic fluid creation system 400 may travel to a pump 512 via, for example, a hose (not shown). In one example, the pump 512 may include a self-priming or non-self-priming pump. In one example, the pump 512 may include a self-priming tank. In one example, the pump 512 may include an end-suction pump wherein the suction created by the pump 512 is axially aligned with respect to a rotation of a drive shaft of the pump 512 and the discharge of the pump 512 is oriented at a 90 degree)(° with respect to the suction. The pump 512 may be selectively activated via the electrical control assembly 402.

The pump 512 may pump the water to a number of filter cartridges included within the fluid filtration assembly 404. In one example, a discharge port of the pump 512 is fluidically coupled to an adapter of the filter cartridges via, for example, a hose (not shown). The filter cartridges of the fluid filtration assembly 404 may filter the water introduced into the cryogenic fluid creation system 400 in order to remove any impurities that may compromise the purity of the to-be-composed cryogenic fluid composition thereby making the cryogenic fluid composition a uniform solution that is free of contaminants. Further, the filter cartridges of the fluid filtration assembly 404 may filter the water in order to remove pathogens (e.g., microorganisms, germs, etc.) that may cause an animal such as a human or livestock to get sick if the to-be-generated cryogenic fluid were to come into contact with the animal and the pathogen remains in the cryogenic fluid.

Once filtered, the water may be pumped by the pump 512 from the filter cartridges of the fluid filtration assembly 404 to an ultraviolet germicidal irradiation (UVGI) assembly 510. The UVGI assembly 510 may include any device that uses ultraviolet light to kill or inactivate microorganisms by destroying nucleic acids and disrupting deoxyribonucleic acid (DNA), leaving the microorganisms unable to perform vital cellular functions. In one example, the UVGI assembly 510 may include an ultraviolet-c (UVC) light-emitting diode (LED) sterilizer. The UVGI assembly 510 may include a power source 534 used to power the light emitting devices within the UVGI assembly 510. Thus, the UVGI assembly 510 may be fluidically coupled to the filter cartridges of the fluid filtration assembly 404 while also being electrically coupled to the power source 534. The power source 534 may be selectively activated by via the electrical control assembly 402. The UVGI assembly 510 may be fluidically coupled to the filter cartridges of the fluid filtration assembly 404 via, for example, a hose (not shown).

Once filtered and sterilized via the filter cartridges of the fluid filtration assembly 404 and the UVGI assembly 510, respectively, the water may be pumped by the pump 512 to a reservoir assembly 506. The reservoir assembly 506 may include any container and a lid to the container. Once retained within the reservoir assembly 506, the water may be mixed with a number of additional chemical substances that may be used to form the cryogenic fluid composition. Examples of the cryogenic fluid composition including non-water chemical compositions are described herein. The additional, non-water chemical substances may be introduced into the reservoir assembly 506 via the lid. In one example, the reservoir assembly 506 may include a mixer to continually mix the cryogenic fluid composition to ensure a homogeneous mixture. The reservoir assembly 506 may be fluidically coupled to the UVGI assembly 510 via, for example, a hose (not shown).

Further, in one example, the reservoir assembly 506 may include a check valve/drain valve assembly 508 coupled between the reservoir assembly 506 and the UVGI assembly 510 to ensure that possibly contaminated fluid (e.g., water) does not enter the reservoir assembly 506. Further, the check valve/drain valve assembly 508 allows for any cryogenic fluid composition contained within the reservoir assembly 506 to be drained from the reservoir assembly 506 as described herein.

The cryogenic fluid composition formed within the reservoir assembly 506 may be held there until introduction to the cryogenic fluid generator assembly 502. The cryogenic fluid composition may be introduced to the cryogenic fluid generator assembly 502 via a port located at the bottom end of the cryogenic fluid generator assembly 502. The cryogenic fluid generator assembly 502 may be fluidically coupled to the reservoir assembly 506 via, for example, a hose (not shown). As will be described in more detail herein, an auger may be used to force the cryogenic fluid composition vertically upwards along an internal chamber of the cryogenic fluid generator assembly 502, expose the cryogenic fluid composition to a decrease in temperature provided by a heat exchanger system of the cryogenic fluid generator assembly 502, and generate the cryogenic fluid. In one example, the auger within the cryogenic fluid generator assembly 502 may be rotated about the vertical axis of the cryogenic fluid generator assembly 502 using a motor 520. In one example, the motor 520 may include a hollow bore gearmotor. In one example, the motor 520 may include a GEARMOTOR F3 high torque alternating current (AC) gear motor with brake (item number: F3S35N20-MV6AWB2) developed and distributed by Brother Industries, Ltd. In one example, the electrical control assembly 402 may be selectively activated via the electrical control assembly 402.

In one example, the motor 520 may also be used to cause the heat exchanger to create a temperature differential between an environment outside the cryogenic fluid generator assembly 502 and the interior of the cryogenic fluid generator assembly 502 such that the cryogenic fluid composition is caused to form into the cryogenic fluid. In this example, the motor 520 may act as a heat pump or compressor that compresses a refrigerant such that the refrigerant may be used to cool the interior of the cryogenic fluid generator assembly 502. The cryogenic fluid may then be dispensed from the cryogenic fluid generator assembly 502 for use as a therapeutic as described herein. The cryogenic fluid may be dispensed or pumped from the cryogenic fluid generator assembly 502 into, for example, the collection reservoir 106 of FIG. 1 via a dispensing spout 532.

The cryogenic fluid generator assembly 502 may include a ball valve 526 coupled to a base of the cryogenic fluid generator assembly 502 via a pipe nipple 524 and an elbow 522. The elbow 522 may couple to a port defined in the base and opening of the ball valve 526 may cause fluid contained within the cryogenic fluid generator assembly 502 to empty. This may be helpful in situations where the fluid must be removed in order to move the cryogenic fluid creation system 400, clean the cryogenic fluid generator assembly 502, or for other purposes. Further, in one example, a spacer 528 may be positioned between the cryogenic fluid generator assembly 502 and the motor 520 in order to allow the motor 520 to mechanically couple to the auger 800 of the cryogenic fluid generator assembly 502 at an intended position along, for example, the entry shaft 810 (e.g., the first neck 1002).

In one example, the heat exchanger of the cryogenic fluid generator assembly 502 may include a resistance temperature detector (RTD) assembly 504 to detect a temperature of a refrigerant used to decrease the internal temperature of the cryogenic fluid generator assembly 502. The RTD assembly 504 may include any sensor whose resistance changes as its temperature changes. The resistance increases as the temperature of the sensor increases. Because an RTD is a passive device, the RTD does not produce an output on its own and a number of external electronic devices may be used to measure the resistance of the sensor by passing a small electrical current through the sensor to generate a voltage. In one example, a 1 milliamp (mA) or less measuring current, with a 5 mA maximum in order to avoid the risk of self-heating. Therefore, the RTD assembly 504 may include an ohmmeter electrically coupled to the electrical control assembly 402 and the RTD assembly 504 to detect the change in resistance. With this information, the electrical control assembly 402 may precisely control the internal temperature of the cryogenic fluid generator assembly 502.

The cryogenic fluid creation system 400 may include a frame assembly 514 including a number of horizontal members and vertical members coupled to one another as depicted, for example, in FIG. 5. The various elements of the cryogenic fluid creation system 400 described herein may be coupled to the frame assembly 514 in order to support these various elements.

Further, a number of housing guards may be included in the cryogenic fluid creation system 400 in order to finish the cryogenic fluid creation system 400, keep the elements of the cryogenic fluid creation system 400 contained within an overall housing and to ensure that those elements are not compromised by users or other influences external to the housing. In one example, the housing guards described herein may be coupled to a number of the horizontal members and vertical members of the frame assembly 514. The housing guards of the cryogenic fluid creation system 400 may include, for example, a top housing guard 406, a front, upper housing guard 408, a front lower right housing guard 410, a front lower left housing guard 412, a left side housing guard 414, a rear housing guard 416, and a right side housing guard 418.

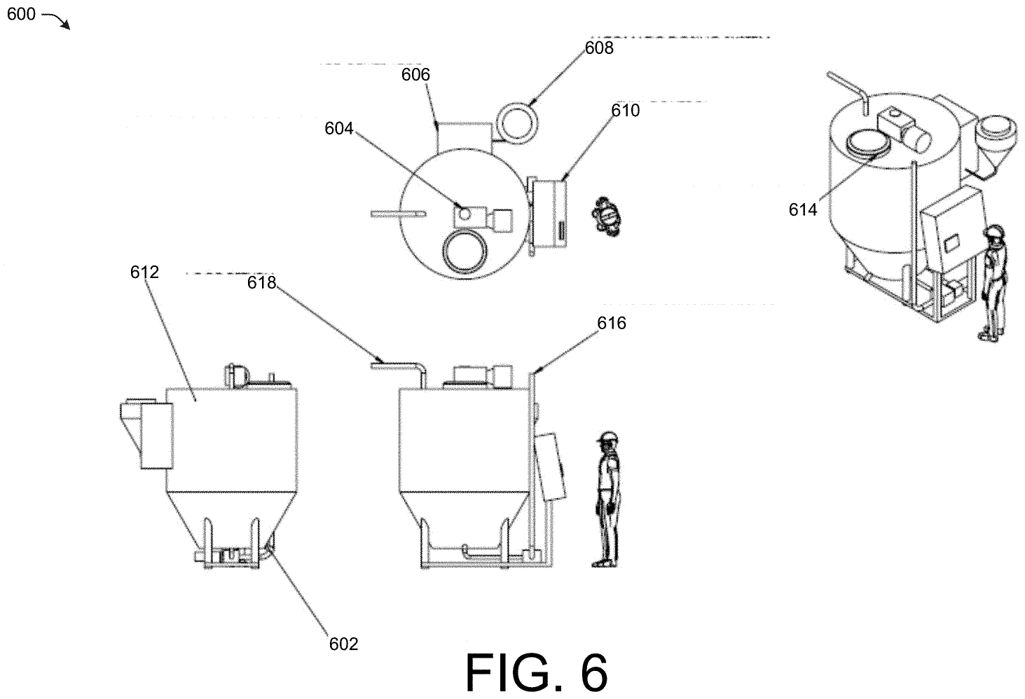

More details regarding the above-described elements of the cryogenic fluid creation system 400 are provided herein. These elements may be of any size, orientation, or shape to allow for the creation of the cryogenic fluid from the cryogenic fluid composition. For example, FIGS. 1 through 3 depict several examples of the cryogenic fluid creation system 400 with varying size, orientation, and shape in order to accommodate for mobility and volume of cryogenic fluid that may be produced. FIG. 6 illustrates a cryogenic fluid creation system 600, according to an example of the principles described herein. The cryogenic fluid creation system 600 of FIG. 6 may be classified as a high volume cryogenic fluid creation system in which the various elements described herein may be enlarged to provide a higher volume of cryogenic fluid. In one example, the cryogenic fluid creation system 600 of FIG. 6 may be deployed as a fixture within a facility such as a medical facility, a sport training facility, a livestock facility, or similar facility in which a number of individuals or livestock may utilize the cryogenic fluid created thereby.

In one example, the cryogenic fluid creation system 600 of FIG. 6 may have a 400 liter (L) to 1,000 L storage and/or processing capacity. Further, although the cryogenic fluid creation system 600 may function in a manner similar to other examples described herein, the cryogenic fluid creation system 600 of FIG. 6 may include a number of additional elements such as an automatic level control system 602 to ensure the cryogenic fluid creation system 600 is kept level with respect to a horizontal plane created by gravity. Further, the cryogenic fluid creation system 600 may include an agitator 604 for agitating the cryogenic fluid to maintain a consistent slurry. Still further, the cryogenic fluid creation system 600 may include a cryogenic fluid generator 606 similar to the cryogenic fluid generator assembly 502 described herein. Even further, the cryogenic fluid creation system 600 may include an automatic dosing system 608 for dispensing the non-water chemical compositions and/or water into the cryogenic fluid generator 606. A fill control system 610 may be used to control the amount of cryogenic fluid within a storage area 612. The fill control system 610 may include the functionality of the electrical control assembly 402 of FIG. 4 described herein. Further, the cryogenic fluid creation system 600 may include an inspection manway 614 to allow for the inspection of the internal portions of the storage area 612 and other elements of the cryogenic fluid creation system 600. Still further, the cryogenic fluid creation system 600 may include a pump 616 to dispense the generated cryogenic fluid to remote areas with, for example, the facility, and a return loop 618 to draw unused cryogenic fluid from the remote area back into the cryogenic fluid creation system 600. Although not depicted the cryogenic fluid creation system 600 may further include an automatic clean in place (CIP) systems to assist in the maintenance of the cryogenic fluid creation system 600.

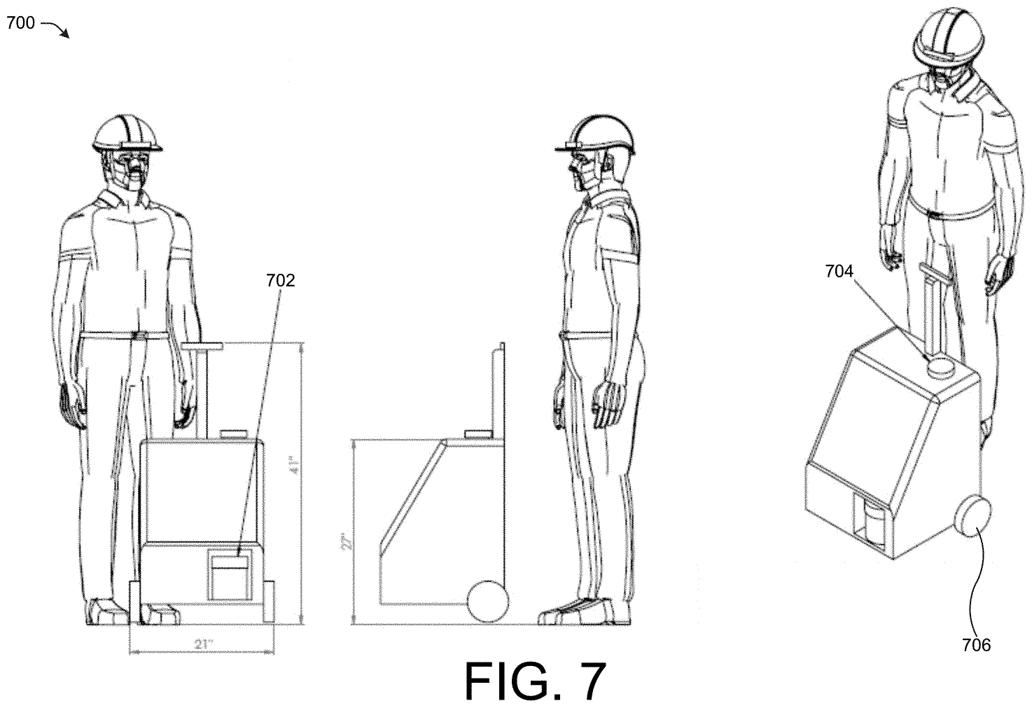

FIG. 7 illustrates a cryogenic fluid creation system 700, according to an example of the principles described herein. The cryogenic fluid creation system 700 includes some or all of the various elements of the other examples of the cryogenic fluid creation systems described herein. The example of FIG. 7 may be used in situations wherein the user, may not be able to be moved to a facility installed example and where the cryogenic fluid creation system 700 may be mobile and brought to the user (e.g., a patient). This allows for the patient to not have to further strain themselves in light of their possible injuries. Further, the cryogenic fluid creation system 700 may include a discharge port 702 to collect the cryogenic fluid generated by the cryogenic fluid creation system 700. Further, the example cryogenic fluid creation system 700 of FIG. 7 may include a fill solution cap 704 that allows for the user to fill a reservoir with water, non-water chemical compositions, the cryogenic fluid composition as a premixed composition, or combinations thereof. Further, the example cryogenic fluid creation system 700 of FIG. 7 may include a number of wheels 706 coupled thereto to allow a user to convey the cryogenic fluid creation system 700 to other locations.

In FIGS. 1, 3, 6 and 7 depict an individual standing next to the various examples of the cryogenic fluid creation systems in order to show relative size of these examples. However, the various examples described herein may take and shape or size as may be fit for an intended purpose.

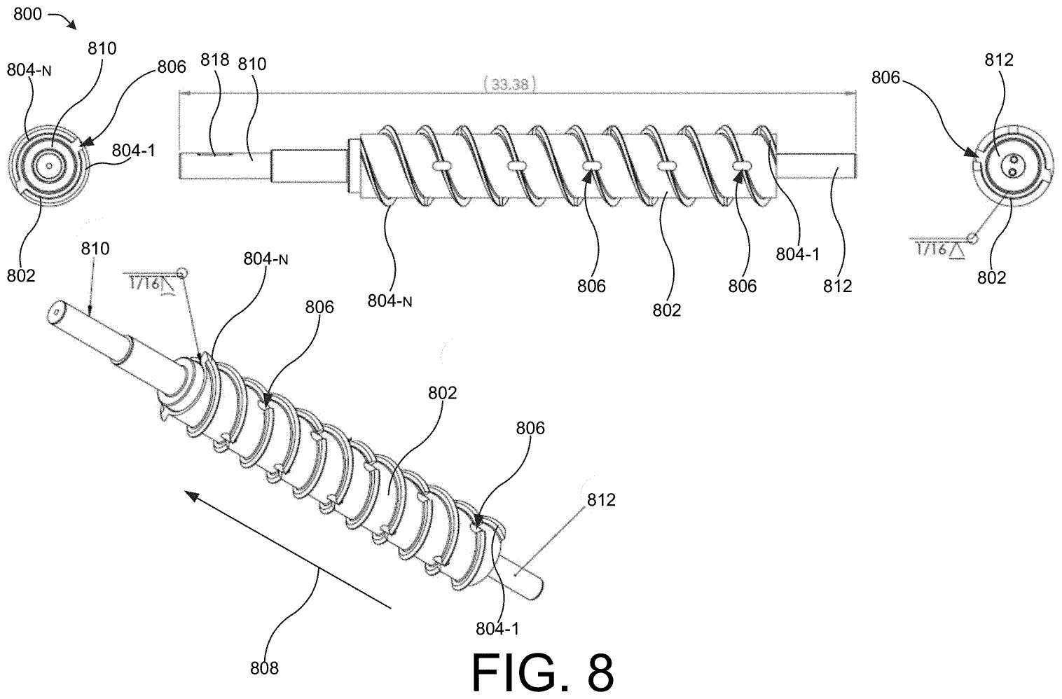

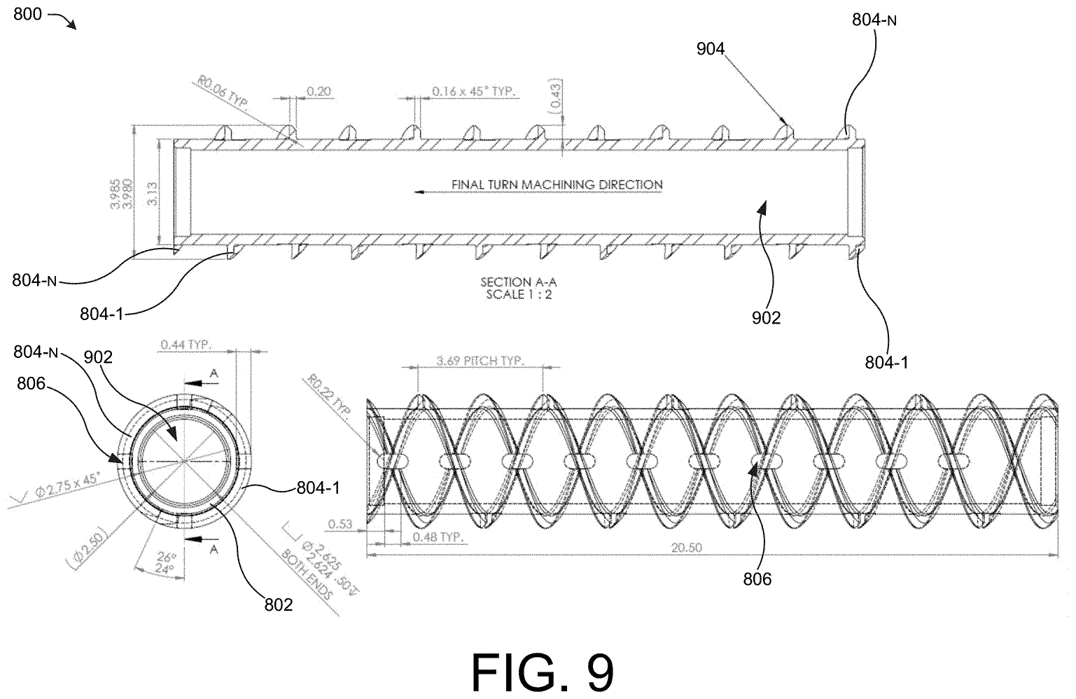

FIG. 8 illustrates an auger 800 for use in a cryogenic fluid creation system, according to an example of the principles described herein. FIG. 9 illustrates a number of different views of the auger 800 of FIG. 8 including a cross-sectional side view, an end-on view, and a side view including threads depicted in solid and in ghost, according to an example of the principles described herein. The auger may be included any rotatable device with a number of helical screw threads. The auger may be housed in a cylindrical housing of, for example, the cryogenic fluid generator assembly 502 to move material through the cylindrical housing. In one example, the auger 800 may also be used to remove material from the inside of the cylindrical housing. The rotation of the auger 800 within the cylindrical housing causes the material to be pulled from the sides of the cylindrical housing and from a first end of the cylindrical housing (e.g., a bottom of the cylindrical housing relative to gravity) to a second end of the cylindrical housing (e.g., a top of the cylindrical housing relative to gravity). In one example, the auger 800 and the cylindrical housing may be used to create pressure in the material being moved through the cylindrical housing by forcing the material to the second end of the cylindrical housing. When used on combination with a cooling coil of a heat exchanger, the auger 800 creates controlled and specific molecular output consistency to form nano-ice crystalline or slurry cryogenic fluid from the cryogenic fluid composition.

The auger 800 may be rotatably coupled to a drive shaft within the cylindrical housing. The auger 800 may include a number of helical threads 804-1, . . . 804-N, where N is any integer greater than or equal to 1 (collectively referred to herein as helical thread(s) 804 unless specifically addressed otherwise) monolithically formed on an auger core 802 of the auger 800. The helical threads 804 may include any inclined plane helically wrapped around the auger core 802 to force the cryogenic fluid upwards before, during, and after the cryogenic fluid composition is frozen. As depicted in FIGS. 8 and 9, the auger 800 may include two helical threads 804 as indicated by the helical threads 804 depicted in solid and in ghost in FIG. 9. However, any number of helical threads 804 may be formed on the auger core 802.

In one example, the helical threads 804 may include a burr 904 on the edge thereof. The burr 904 may include a portion along a width of the helical threads 804 that is angled at approximately 45° with respect to a surface of the auger core 802. In one example, the burr 904 may include a portion along a width of the helical threads 804 that is angled at approximately between 30° and 55° with respect to the surface of the auger core 802. By including a different angle along the length of the helical threads 804 allows for the helical threads 804 may scrape cooling and freezing cryogenic fluid composition from an interior wall of a cylindrical housing in which the auger 800 is housed and mechanically rotated. As the cryogenic fluid composition is introduced into the cylindrical housing and is cooled by a heat exchanger, the cryogenic fluid composition begins to freeze and generate nano-sized frozen particles once being scraped from the edge of the cylindrical housing by the burrs 904. Once scraped off the interior of the cylindrical housing, the nano-sized frozen particles of the cryogenic fluid may be pushed up the length of the auger 800 and out of the cryogenic fluid creation system.

As indicated in FIG. 8, arrow 808 indicates the direction of the force of gravity as the auger 800 is oriented in the cryogenic fluid generator assembly 502. The auger 800 may further include a number of port holes 806 defined in the auger core 802 to allow for unfrozen cryogenic fluid composition to fall to the bottom of the cylindrical housing in which the auger 800 is housed in order to allow the unfrozen cryogenic fluid composition to undergo further chilling as it is raised along the length of the auger 800 via the helical threads 804 until the cryogenic fluid composition is eventually frozen into the cryogenic fluid. In one example, the auger 800 may include port holes 806 defined along each quarter of a circumference of the auger core 802. The auger 800 may include other features than those described herein in addition to and/or in place of these features. However, the auger 800 functions to move the cryogenic fluid composition through the cryogenic fluid creation system 100, produce frozen cryogenic fluid, and push the frozen cryogenic fluid out the dispensing spout 532.

In one example, the auger 800 as driven by the motor 520 may produce a linear scraping speed of between 15 millimeters per minute (mm/min) and 3 mm/min at a gap ranging from 0.005 in. to 0.015 in. due to the rotational speed (e.g., revolutions per minute (RPM)) and relative size of the helical threads 804. Due to the rotational speeds, relative sizes of the auger helix, and closeness (e.g., small gap) of the auger 800 relative to an interior wall (e.g., interior wall 1406 of FIG. 14) small features may be broken off of the interior wall through, for example, shear forces, into molecular, nanoparticles (e.g., nano-ice). Further, in one example, the cryogenic fluid composition as frozen may be stuck or adhered to the interior wall by a prescribed roughness.

Further, this speed can be computer controlled by measuring the amperage torque on the motor 520 by, for example, the electrical control assembly 402 to control the ideal rate of cryogenic fluid production. As the cryogenic fluid generator assembly 502 gets colder and reaches steady-state producing more cryogenic fluid, the linear speed of the auger 800 may be sped-up. The ability to control the rotation speed of the auger 800 provides for a more effective production of the cryogenic fluid if the solution percentage of the cryogenic fluid composition is not known, exact, or varies due to mixing. This torque sensing may be automatically adjusted as a percent solution of the cryogenic fluid composition goes from a very concentrated mix (e.g., a relatively lower freezing temperature such as, for example, 24°) to a more diluted one (e.g., a relatively higher freezing temperature such as, for example, 31°). The torque sensing will also adjust the relative viscosity or ice fraction of the cryogenic fluid composition. In one example, the cryogenic fluid, once frozen, may have between 10% and 50% ice/water in order to retain therapeutic benefits.

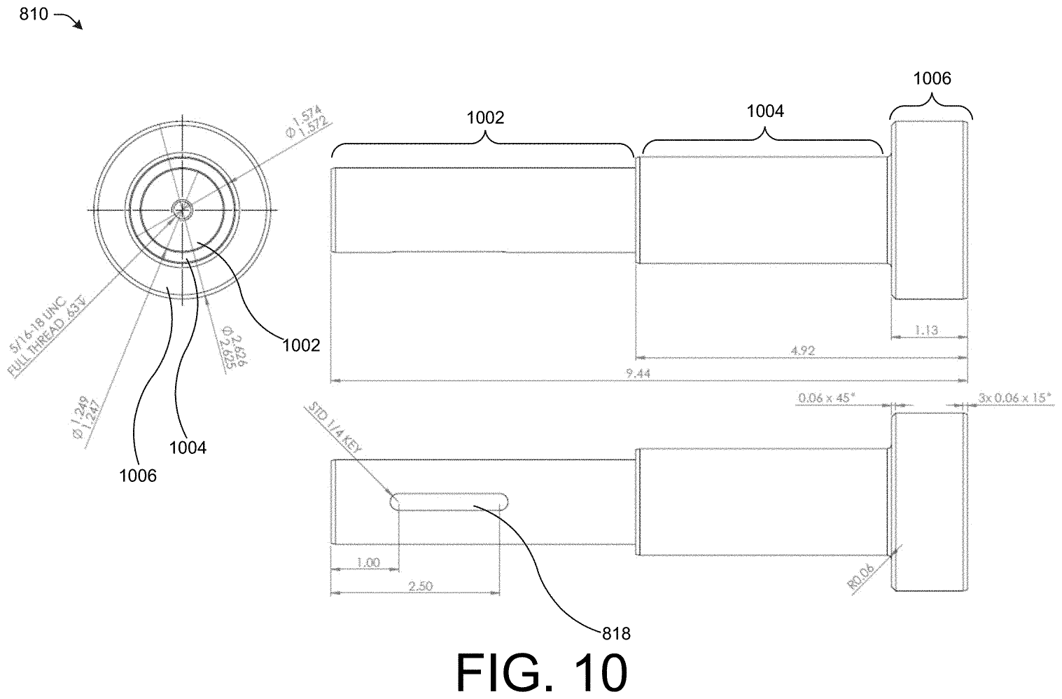

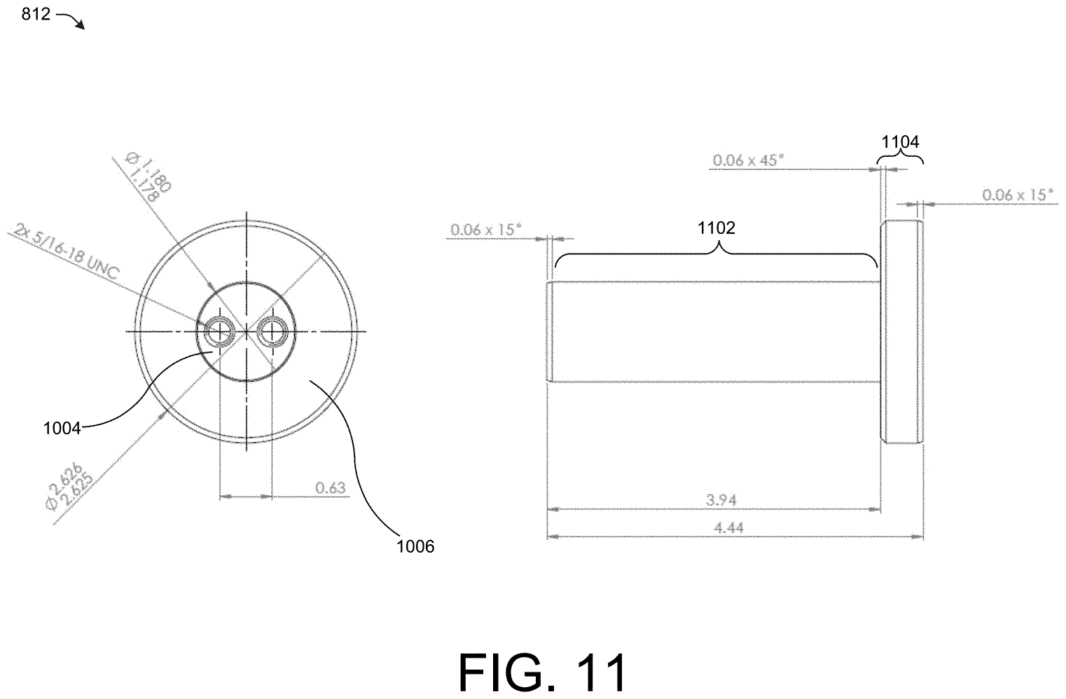

FIG. 10 illustrates an entry shaft 810 coupled to a top of the auger 800 of FIGS. 8 and 9, according to an example of the principles described herein. FIG. 11 illustrates an exit shaft 812 coupled to a bottom of the auger 800 of FIGS. 8 and 9, according to an example of the principles described herein. In one example, the auger 800 may be hollow by defining a void 902 inside the auger core 802. As depicted in FIGS. 8-11, the void 902 may be fluidically couped to the port holes 806 defined in the auger core 802 to allow for unfrozen cryogenic fluid composition to flow into and out of the void 902 and move freely along the length of the auger 800.

The entry shaft 810 may include a key seat 818. The key seat 818 may be configured to receive a key in order to couple the entry shaft 810 to a keyway of a drive shaft of the motor 520. In this manner, the entry shaft 810 of the auger 800 may be mechanically coupled to the motor 520 so that the motor may rotate the auger 800. Further, the entry shaft 810 may include a first neck 1002, a second neck 1004, and a base 1006. The exit shaft 812 may include a first neck 1102 and a base 1104. The first neck 1002, second neck 1004, base 1006, first neck 1102, and base 1104 may be used as structures to which bearings or other elements may mechanically couple to the auger 800 to support the auger 800 as it rotates.

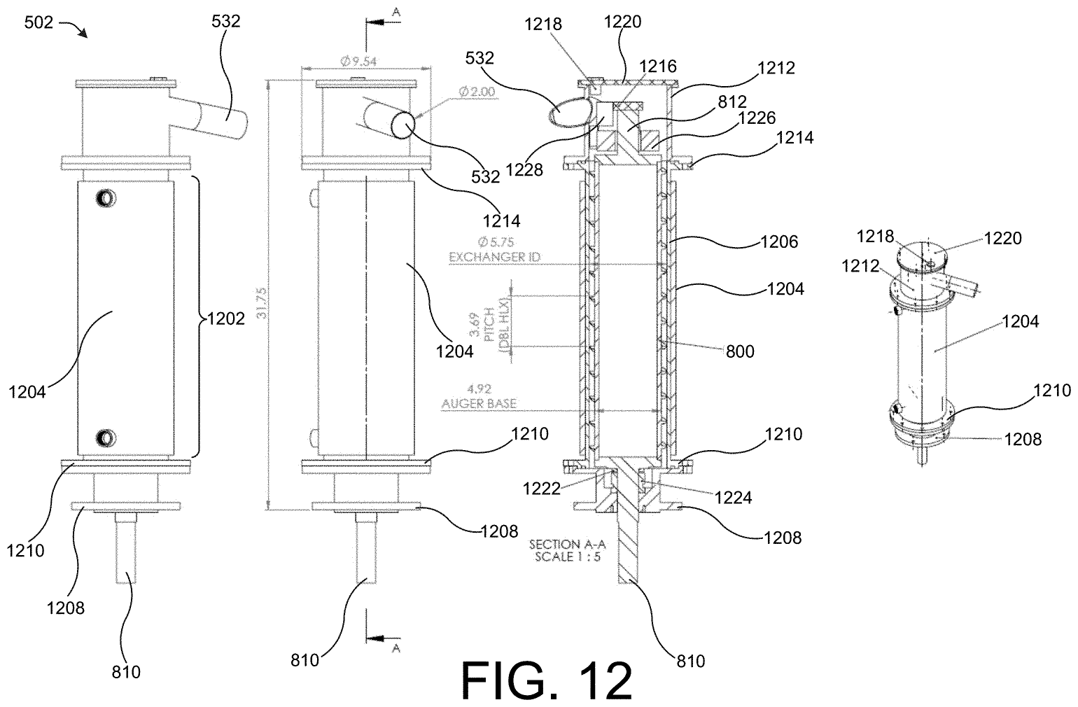

FIG. 12 illustrates a cryogenic fluid generator assembly 502 of a cryogenic fluid creation system, according to an example of the principles described herein. The cryogenic fluid creation system 100, 200, 300, 400, 600, 700 of FIGS. 1-7 may incorporate the cryogenic fluid generator assembly 502 of FIG. 12. The cryogenic fluid generator assembly 502 may include the auger 800 of FIGS. 8 and 9. Further, the cryogenic fluid generator assembly 502 may include a heat exchanger 1202 including a shell 1204 of the heat exchanger 1202, a core 1206 of the heat exchanger 1202, a base of the heat exchanger, a bottom flange 1210 of the heat exchanger, and a top flange 1214 of the heat exchanger, among other elements described herein.

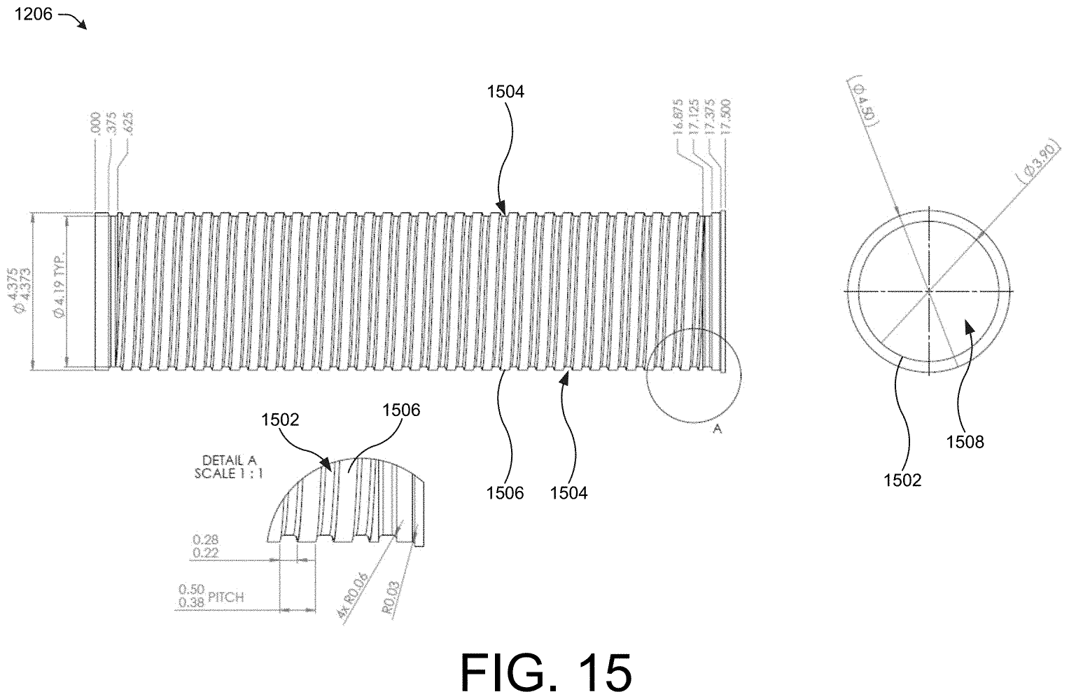

As depicted in FIG. 12, the core 1206 may house the auger 800. FIG. 15 illustrates the core 1206 of the heat exchanger 1202 for use in a cryogenic fluid creation system, according to an example of the principles described herein. The core 1206 provides a surface against which the burrs 904 of the helical threads 804 may scrape the frozen cryogenic fluid once the core 1206 is brought to a temperature by the heat exchanger 1202 at which the cryogenic fluid composition may freeze. Thus, the core 1206 may include an interior wall 1502 against which the burrs 904 may scrape. In one example, the interior wall 1502 may be smooth. In one example, the interior wall 1502 may be textured to promote crystal formation. The exterior of the core 1206 may include a number of channels 1504 defined in the outer surface 1506. The channels 1504 are configured to carry compressed refrigerant that may be used to bring the temperature of the interior of the core 1206 including the temperature of the interior wall 1502 of the core 1206, the auger 800, and the cryogenic fluid composition to be frozen therein. The shell 1204 may be placed around the core 1206 in order to close the channel 1504 with an interior wall of the shell 1204. In this manner, the compressed refrigerant may be contained between the shell 1204 and the core 1206.

The base 1208 may be positioned at the end of the cryogenic fluid generator assembly 502 at which the entry shaft 810 of the auger 800 is located. In this manner, the base 1208 may be positioned at the bottom of the cryogenic fluid generator assembly 502. The base 1208 may be coupled to a bottom flange 1210. The bottom flange 1210 may be coupled to the core 1206 of the heat exchanger 1202 via any fastening device or method such as, for example, an engineering fit. As used in the present specification and in the appended claims, the term “engineering fit” is meant to be understood broadly as any engineering fit such as, for example, a clearance fit (e.g., one of a loose running fit, a free running fit, a close running fit, a sliding fit, and a location fit), a transition fit (e.g., one of a similar fit, and a fixed fit), and an interference fit (e.g., one of a press fit, a driving fit, and a forced fit). In this manner, the bottom flange 1210 may be coupled to the core 1206 via an engineering fit. Further, the base 1208 may be coupled to a bottom flange 1210 via any fastening device or method such as, for example, a number of bolts, nuts, screws, or other types of fasteners. The entry shaft 810 of the auger 800 may extend through and seat within the base 1208 and the bottom flange 1210 in order to allow the entry shaft 810 to couple to the motor 520.

The top flange 1214 may be coupled to the core 1206 of the heat exchanger 1202 via any fastening device or method such as, for example, an engineering fit. A discharge top 1212 may be coupled to the top flange 1214 via any fastening device or method such as, for example, a number of bolts, nuts, screws, or other types of fasteners. The discharge top may include a cap 1220 to close the discharge top 1212. An entry cap 1218 may be included in the cap 1220 to allow for access to the interior of the discharge top 1212.

The exit shaft 812 of the auger 800 may extend through and seat within the top flange 1214 and the discharge top 1212 in order to allow for the rotation of the auger 800 to rotate a discharge impeller 1216. The discharge impeller 1216 is used to convey the cryogenic fluid out of the cryogenic fluid generator assembly 502 through the dispensing spout 532. more details regarding the elements of the cryogenic fluid generator assembly 502 are provided herein.

In one example, a shaft seal washer 1222 may be located between a face of the base 1006 of the entry shaft 810 and a mechanical shaft seal 1224. The mechanical shaft seal 1224 may be seated between the base 1208 of the entry shaft 810 and the base 1208 coupled to the bottom flange 1210. The mechanical shaft seal 1224 may include any device that is able to remain stationary with respect to the rotating entry shaft 810, allow the entry shaft 810 to rotate, and ensure that any fluids or contaminants do not move past the mechanical shaft seal 1224 and/or the base 1208 to the motor 520. In one example, the mechanical shaft seal 1224 may include an EA560 mechanical shaft seal developed and distributed by Eagle Burgmann Industries, Ltd.

In one example, the discharge top 1212 may include a mount 1226 that serves to support the exit shaft 812 with in the discharge top 1212 while allowing the exit shaft 812 to freely rotate. The discharge impeller 1216 may be coupled to an end of the exit shaft 812 in order for the rotation of the exit shaft 812 and auger 800 to impart rotational force to the discharge impeller 1216. In one example, the discharge impeller 1216 may be coupled to an end of the exit shaft 812 via a set screw, a key and key seat pair, or other device or method that locks the rotation of the discharge impeller 1216 with the rotation of the exit shaft 812. The discharge impeller 1216 may include a push face 1228 to push the frozen cryogenic fluid moved up the cryogenic fluid generator assembly 502 by the auger 800 and into the discharge top 1212, out of the discharge top 1212 via the dispensing spout 532. Once pushed out of the discharge top 1212 via the dispensing spout 532, the cryogenic fluid may be caused to be collected in the collection reservoir 106.

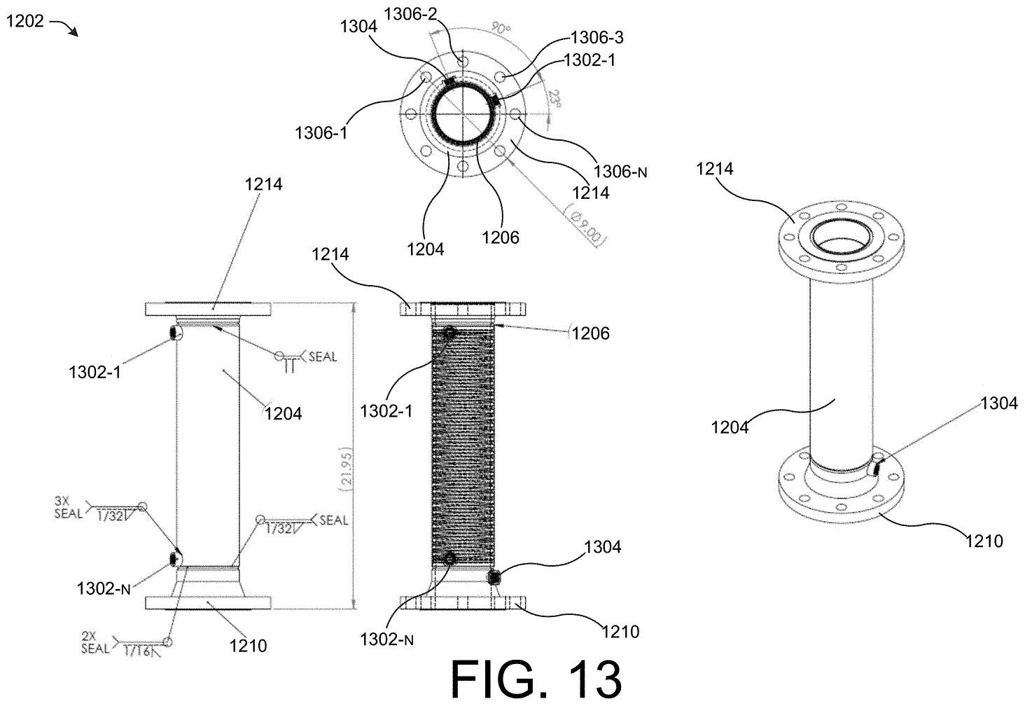

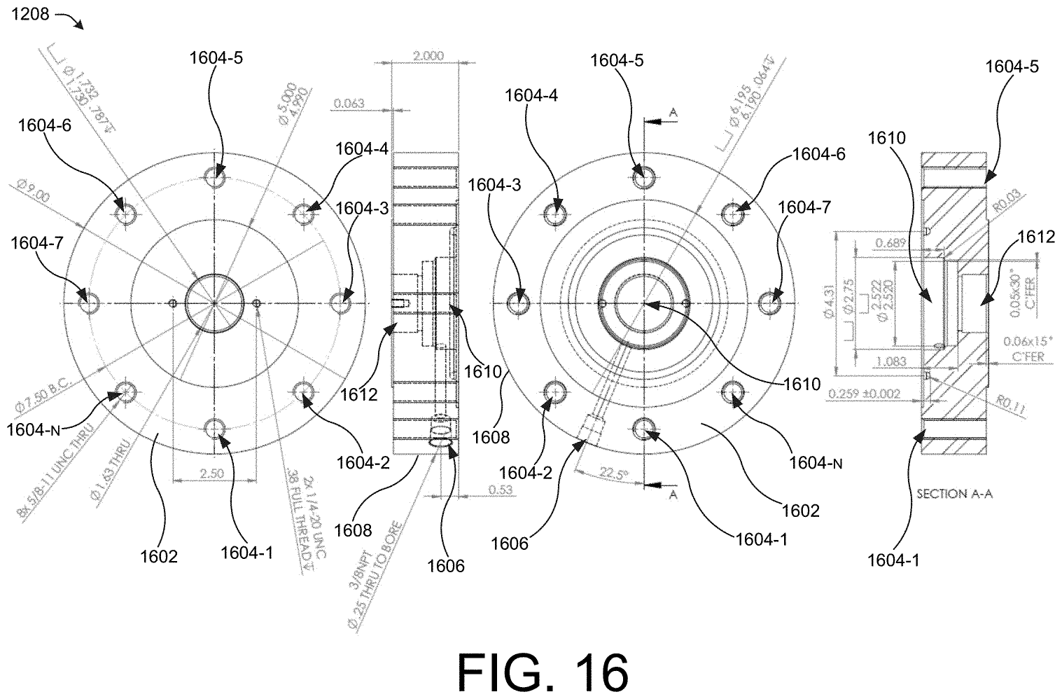

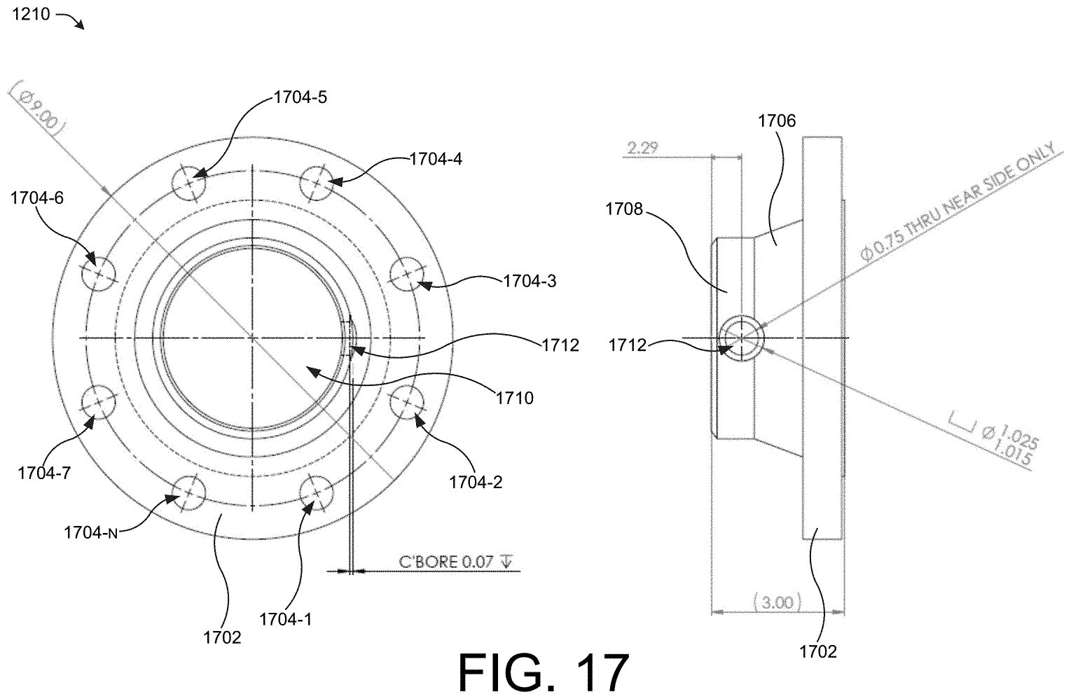

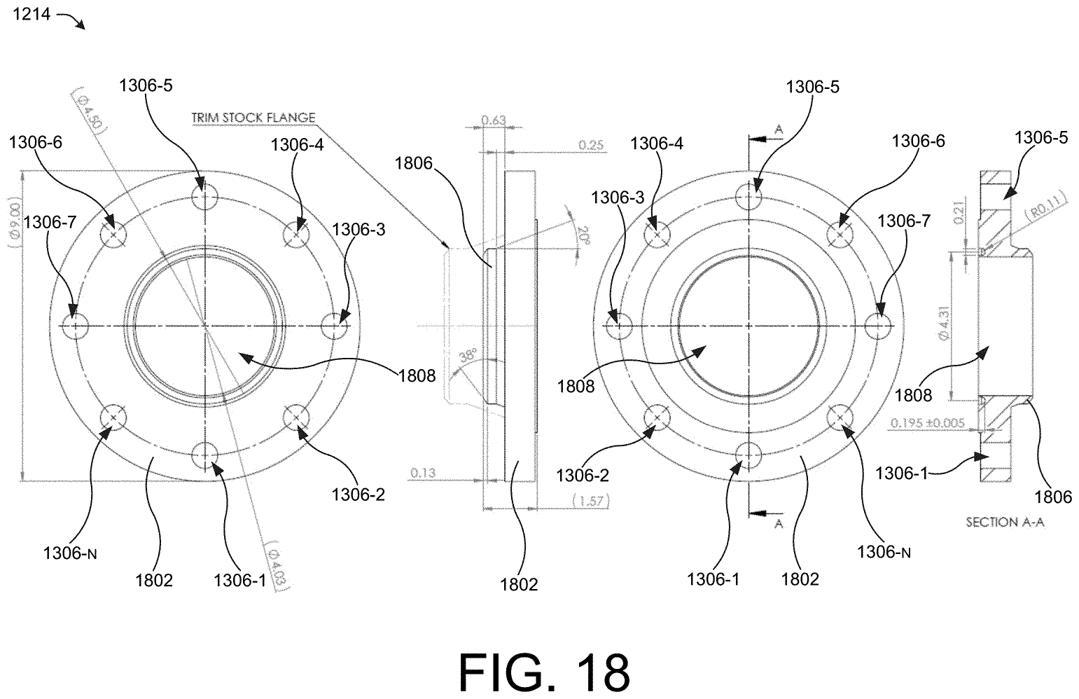

FIG. 13 illustrates a heat exchanger 1202 used in a cryogenic fluid creation system, according to an example of the principles described herein. FIG. 14 illustrates a shell of a heat exchanger 1202 use in a cryogenic fluid creation system, according to an example of the principles described herein. FIG. 15 illustrates a core of a heat exchanger 1202 use in a cryogenic fluid creation system, according to an example of the principles described herein. FIG. 16 illustrates a base of a heat exchanger 1202 for use in a cryogenic fluid creation system, according to an example of the principles described herein. FIG. 17 illustrates a bottom flange of a heat exchanger 1202 for use in a cryogenic fluid creation system, according to an example of the principles described herein. FIG. 18 illustrates a top flange of a heat exchanger 1202 for use in a cryogenic fluid creation system, according to an example of the principles described herein. The heat exchanger 1202 as depicted in FIG. 13 may include the shell 1204, the core 1206, the bottom flange 1210, and the top flange 1214.

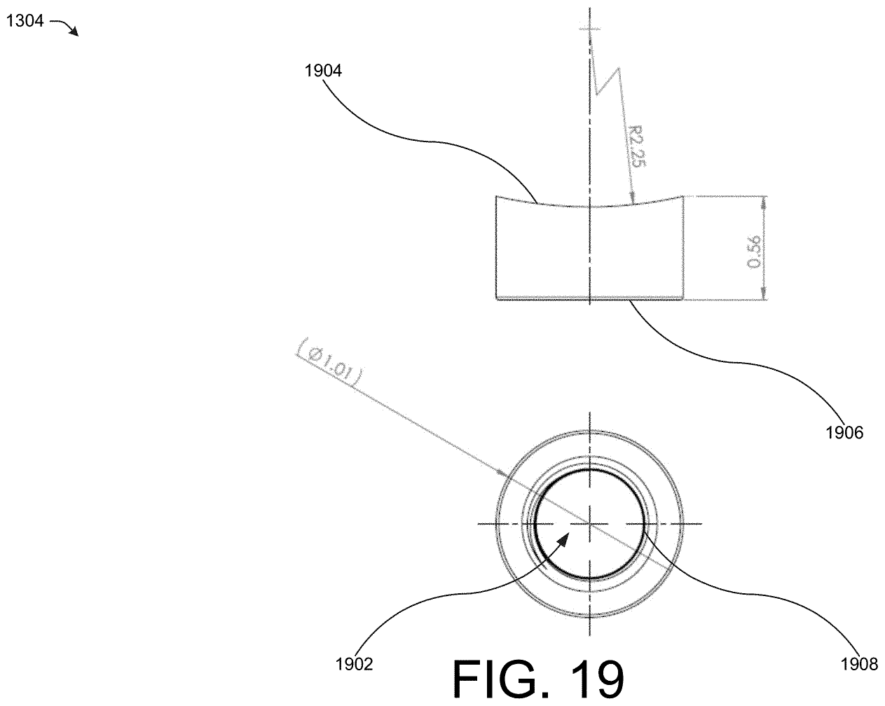

Further, the bottom flange 1210 may include a flange half coupling 1304. The half couplings described herein may include the half coupling described in connection with FIG. 19. The flange half coupling 1304 is used to couple the heat exchanger 1202 of the cryogenic fluid generator assembly 502 with the reservoir assembly 506 via, for example, a hose (not shown) coupled between a discharge port of the reservoir assembly 506 and the flange half coupling 1304.

The shell 1204 of the heat exchanger 1202 may also include a number of shell half couplings 1302-1, . . . 1302-N, where N is any integer greater than or equal to 1 (collectively referred to herein as shell half coupling(s) 1302 unless specifically addressed otherwise). The shell half couplings 1302 provide a fluid pathway through which the compressed refrigerant may travel to and from the core 1206 in order to chill the interior of the core 1206, the auger 800 and other elements of the heat exchanger 1202. In one example, a first hose (not shown) may couple the shell half coupling 1302-N to a refrigerant compressor (not show) so that the compressed refrigerant may be introduced into the core 1206. Further, a second hose (not shown) may couple the shell half coupling 1302-1 to the refrigerant compressor (not show) so that the decompressed refrigerant may be removed from the core 1206 and returned back to the refrigerant compressor.

In one example, the flange half coupling 1304 may be oriented at approximately 90° with respect to the shell half couplings 1302 as depicted in the top view of the heat exchanger 1202 of FIG. 13. Orientation of the flange half coupling 1304 and the shell half couplings 1302 in this manner allows for the hoses and other devices to clear one another as they are coupled to the cryogenic fluid generator assembly 502. Further, in one example, the flange half coupling 1304 and shell half couplings 1302 may be oriented between a number of top flange bolt holes 1306-1, 1306-2, 1306-3, . . . 1306-N, where N is any integer greater than or equal to 1 (collectively referred to herein as top flange bolt holes(s) 1306 unless specifically addressed otherwise). In one example, the bolt holes defined in the top flange 1214 may vertically align with bolt holes defined in the bottom flange 1210. Further, by way of example, the flange half coupling 1304 may be vertically oriented between a first top flange bolt hole 1306-1 and a second top flange bolt hole 1306-2. Similarly, in one example, the shell half couplings 1302 may be vertically oriented between a third top flange bolt hole 1306-3 and a fourth top flange bolt hole 1306-N. The vertical orientation of the flange half coupling 1304 and the flange half coupling 1304 between the top flange bolt holes 1306 may allow for bolts and nuts to be accessed when coupling the bottom flange 1210 to the base 1208 and the top flange 1214 to the discharge top 1212.

Turning to FIG. 14, the shell 1204 of the heat exchanger 1202 may include the shell half couplings 1302 may be defined in a body 1402. In order to accommodate for the fitting of the core 1206 and the auger 800 within the shell 1204, the shell 1204 may have a cylindrical shape with an internal void 1404. The internal void 1404 defines an interior wall 1406 that, when installed, abuts the outer surface 1506 of the core 1206.

Turning to FIG. 15, the core 1206 may also include a void 1508. The auger 800 may fit within the void 1508 of the core 1206. The auger 800 and/or the core 1206 including the interior wall 1502 and the void 1508 may be dimensioned such that the distance between the helical threads 804 and burrs 904 of the auger 800 and the interior wall 1502 of the core 1206 is between 0.005 in. to 0.015 in. With this clearance between these elements, the nanoparticles of ice may be formed by freezing the cryogenic fluid composition into the cryogenic fluid and allowing the burrs 904 of the helical threads 804 of the auger 800 to scrape the nanoparticles from the interior wall 1502 of the core 1206.

Turning to FIG. 16, the base 1208 may include a disc-shaped body 1602 with a number of base bolt holes 1604-1, 1604-2, 1604-3, 1604-4, 1604-5, 1604-6, 1604-7, 1604-N, where N is any integer greater than or equal to 1 (collectively referred to herein as base bolt holes(s) 1604 unless specifically addressed otherwise) defined therein. The bolt holes 1604 may be defined in the disc-shaped body 1602 of the base 1208 in an identical orientation and spacing as other bolt holes defined in the bottom flange 1210 so that the base 1208 and the bottom flange 1210 may be coupled together via a number of bolts, nuts, etc.

As depicted in FIG. 16, the base 1208 may include an aperture 1606 defined from an edge 1608 of the base 1208 through to a bore 1610. The aperture 1606 may be defined in the base 1208 perpendicular to a direction at which the base bolt holes 1604 are defined in the base 1208. Further, the bore 1610 may be defined in the base 1208 parallel to a direction at which the base bolt holes 1604 are defined in the base 1208. The aperture 1606 may be threaded to allow for a set screw (not shown) to be engaged therewith. The set screw may be used to ensure that the mechanical shaft seal 1224 seated within a seat of the bore 1610 and around the entry shaft 810 does not rotate with the entry shaft 810 but remains stationary.

An entry shaft aperture 1612 may also be defined in a side of the base 1208 opposite the bore 1610. The entry shaft aperture 1612 may be dimensioned to allow the base 1006 of the entry shaft to seat within the base 1208.

Turning to FIG. 17, the base 1208 may couple either directly or indirectly to the bottom flange 1210. The bottom flange 1210 may include a disc-shaped body 1702, a conical-shaped extension 1706 coupled to and extending from the disc-shaped body 1702, and a ring 1708 coupled to and extending from the conical-shaped extension 1706. In one example, the disc-shaped body 1702, the conical-shaped extension 1706, and the ring 1708 may be monolithically formed as a single piece such as through machining, welding, etc. The ring 1708 couples to the core 1206 and/or the shell 1204 of the heat exchanger 1202 and serves to further house the auger 800.

The disc-shaped body 1702 may include a number of bottom flange bolt holes 1704-1, 1704-2, 1704-3, 1704-4, 1704-5, 1704-6, 1704-7, 1704-N, where N is any integer greater than or equal to 1 (collectively referred to herein as bottom flange bolt holes(s) 1704 unless specifically addressed otherwise) defined therein. The bottom flange bolt holes 1704 of the bottom flange 1210 may align with the base bolt holes 1604 of the base 1208 in order to allow bolts to be extended through both the bottom flange bolt holes 1704 of the bottom flange 1210 and the base bolt holes 1604 of the base 1208. In this manner, the bottom flange 1210 may be coupled to the base 1208.

The bottom flange 1210 may include a bore 1710 defined therein. Further, an injection port 1712 may be defined in the ring 1708 to allow for the cryogenic fluid composition provided from the reservoir assembly 506 to enter the bore 1710 and begin the freezing process described herein. The flange half coupling 1304 described herein in connection with the heat exchanger 1202 may be coupled to the bottom flange 1210 via the injection port 1712. Further, the RTD assembly 504 may be coupled to the flange half coupling 1304.

Turning to FIG. 18, the top flange 1214 couples to the core 1206 and/or the shell 1204 of the heat exchanger 1202 and serves to further house the auger 800. The top flange 1214 may include a disc-shaped body 1802. The disc-shaped body 1802 a may include the top flange bolt holes 1306 defined therein. The top flange 1214 may further include a ring 1806 coupled to and extending from the disc-shaped body 1802. In one example, the disc-shaped body 1802 and the ring 1806 may be monolithically formed as a single piece such as through machining, welding, etc. The ring 1806 couples to the core 1206 and/or the shell 1204 of the heat exchanger 1202 and serves to further house the auger 800. Further, the top flange 1214 may include a bore 1808 defined therein through which the exit shaft 812 extends.

FIG. 19 illustrates a flange half coupling 1304 including NPT threads for coupling pressurized fluid lines within a cryogenic fluid creation system 400, according to an example of the principles described herein. The flange half coupling 1304, as mentioned above, may be coupled to the bottom flange 1210 via the injection port 1712. The flange half coupling 1304 may be used to couple the heat exchanger 1202 of the cryogenic fluid generator assembly 502 with the reservoir assembly 506 via, for example, a hose (not shown) coupled between a discharge port of the reservoir assembly 506 and the flange half coupling 1304. The flange half coupling 1304 may include a bore 1902 defined therein to allow the cryogenic fluid composition to enter the heat exchanger 1202 of the cryogenic fluid generator assembly 502 and interface with the internal surface of the core 1206 and the auger 800. Further, the flange half coupling 1304 may include a curved side 1904 that allows the flange half coupling 1304 to be coupled to the curved surface of the ring 1708 and/or the conical-shaped extension 1706 of the bottom flange 1210. A straight edge 1906 may be included on a side of the flange half coupling 1304 opposite the curved side 1904. A number of threads 1908 may be formed on an interior surface of the bore 1902 to allow for the RTD assembly 504, hoses, or other devices described herein.

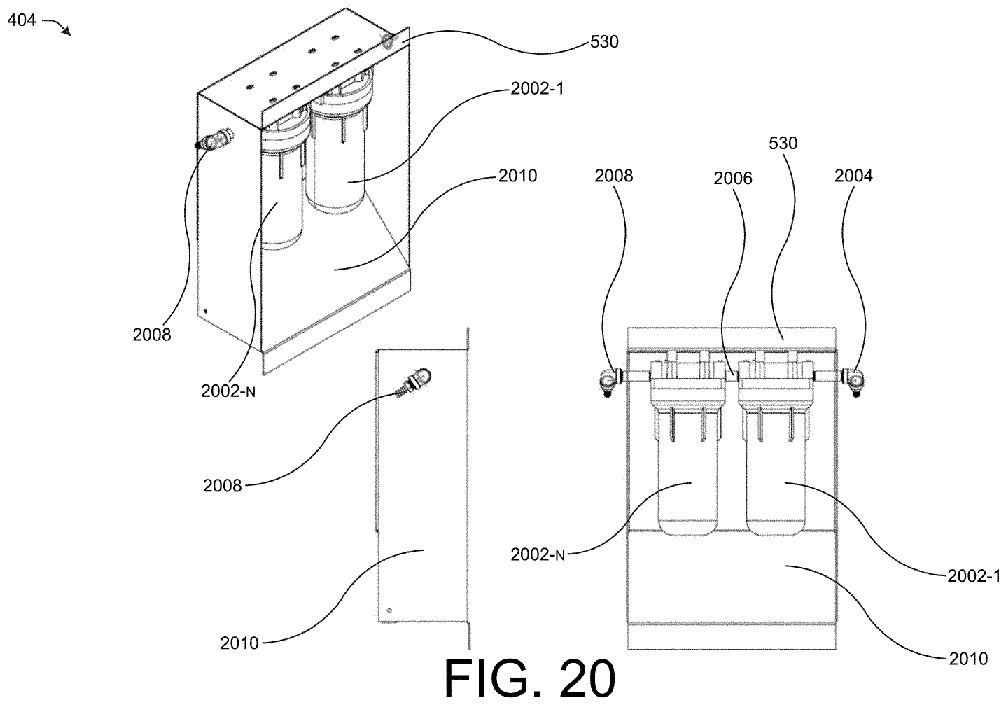

FIG. 20 illustrates a fluid filtration assembly 404 of a cryogenic fluid creation system 400, according to an example of the principles described herein. The fluid filtration assembly 404 may include a water filter housing 2010 to house a number of fluid filter devices 2002-1, . . . , 2002-N, where N is any integer greater than or equal to 1 (collectively referred to herein as fluid filter device(s) 2002 unless specifically addressed otherwise). The fluid filter devices 2002 may be used to filter any fluid used within the cryogenic fluid creation system 400 such as, for example, water.

The water filter housing 2010 may be coupled to a number of elements of the frame assembly 514 via the corner machine bracket 530. Any number of corner machine brackets 530 may be used to couple the water filter housing 2010 to the frame assembly 514.

The fluid filtration assembly 404 may include an inlet port 2004 to allow for the fluid to enter the fluid filter devices 2002. In one example, the fluid filtration assembly 404 receives the fluid from the pump 512 via the inlet port 20. A number of connecting pipes 2006 may be included to fluidically couple the fluid filter devices 2002. Further, the fluid filtration assembly 404 may include an outlet port 2008 to allow the filtered fluid to proceed to, for example, the UVGI assembly 510. In one example, the inlet port 2004 and the outlet port 2008 of the fluid filtration assembly 404 may include barbed hose adapters to couple hoses other elements of the cryogenic fluid creation system 400.

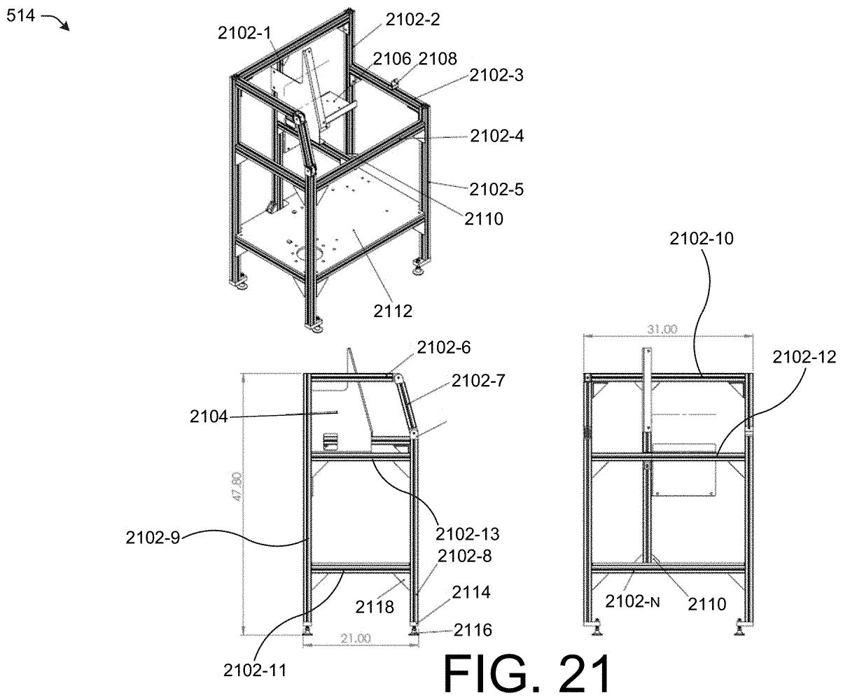

FIG. 21 illustrates a frame assembly 514 of a cryogenic fluid creation system 400, according to an example of the principles described herein. The frame assembly may include, for example, a number of slotted frame pieces 2102-1, 2102-2, 2102-3, 2102-4, 2102-5, 2102-6, 2102-7, 2102-8, 2102-9, 2102-10, 2102-11, 2102-12, 2102-13, 2102-N, where N is any integer greater than or equal to 1 (collectively referred to herein as slotted from piece(s) 2102 unless specifically addressed otherwise). The slotted frame pieces 2102 assist in creating a shape of the cryogenic fluid creation system 400 and in allowing various elements to be mounted to the cryogenic fluid creation system 400 as depicted in, for example, FIGS. 4 and 5.

The frame assembly 514 may further include an electrical box mount 2104 to mount the electrical control assembly 402, a reservoir shelf 2106 to mount the reservoir assembly 506, and other mounts, shelves, and brackets to couple the various elements of the cryogenic fluid creation system 400 to the frame assembly 514. Further, in order to assist in securing the various elements of the cryogenic fluid creation system 400 to the frame assembly 514, a number of brackets 2108 may couple to the slotted frame pieces 2102. For example, in FIG. 21, the bracket 2108 may be an electrical box bracket that couples the electrical control assembly 402 to the slotted frame pieces 2102. Any number of elements within the cryogenic fluid creation system 400 may utilize a bracket 2108 to couple those elements to the slotted frame pieces 2102.

To support the slotted frame pieces 2102 and the weight placed on the slotted frame pieces 2102, a number of corner gussets 2110 may be placed at the connections between slotted frame pieces 2102. In one example, the corner gussets 2110 may be dimensioned and configured to engage with the slots of the slotted frame pieces 2102 so that adjacent slotted frame pieces 2102 are mechanically coupled to one another. Further, inclusion of the corner gussets 2110 causes adjacent slotted frame pieces 2102 to be strengthen and bear relatively heavier loads as compared to not employing the corner gussets 2110. In one example, the frame assembly 514 may further include a number of welded corner angles 2118 to provide further support between horizontally oriented and vertically oriented slotted frame pieces 2102.

The frame assembly 514 may further include a base plate 2112. In one example, the base plate 2112 may include a number of apertures to assist in coupling the various elements of the cryogenic fluid creation system 400 to the frame assembly 514. For example, an aperture may be defined in the base plate 2112 to position the motor 520 under the cryogenic fluid generator assembly 502 and couple at least one of the motor 520 and the cryogenic fluid generator assembly 502 to the base plate 2112. The base plate 2112 may further include a number of smaller apertures to allow for bolts to extend therethrough and couple the various elements of the cryogenic fluid creation system 400 to the base plate 2112.

The frame assembly 514 may further include a number of leveling anchor plates 2114 and adjustable swivel legs 2116 coupled to a number of horizontally oriented slotted frame pieces 2102. The leveling anchor plates 2114 and adjustable swivel legs 2116 may ensure that the frame assembly 514 is level with respect to a surface on which the cryogenic fluid creation system 400 sits. In one example, a number of wheels or casters may be coupled to the horizontally oriented slotted frame pieces 2102 to allow for the cryogenic fluid creation system 400 to be moved.

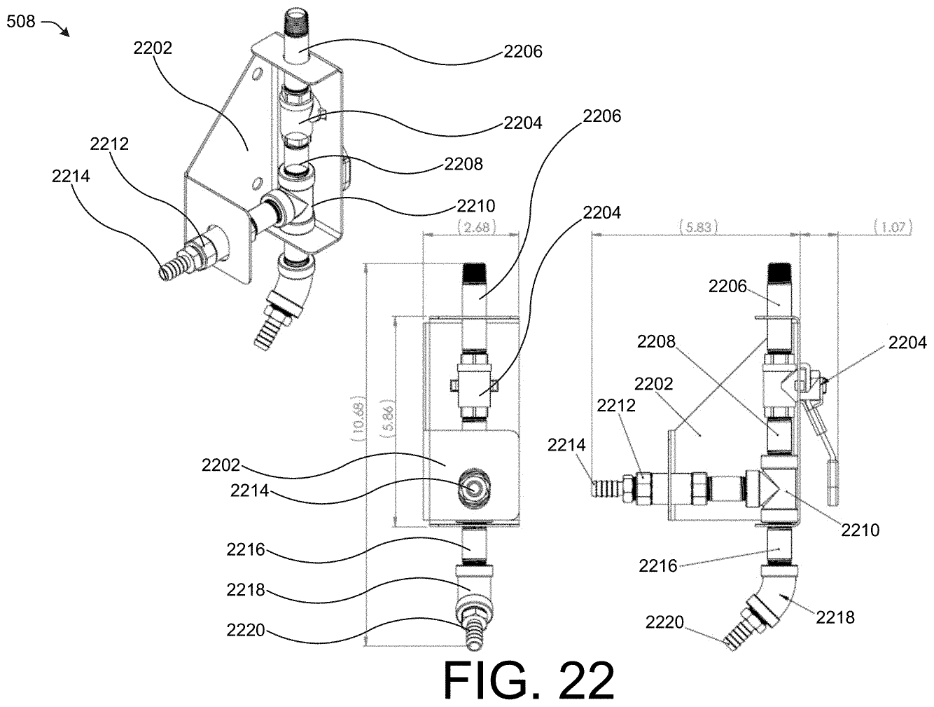

FIG. 22 illustrates a check valve/drain valve assembly 508 of a cryogenic fluid creation system 400, according to an example of the principles described herein. The check valve/drain valve assembly 508 may include a valve bracket 2202 to secure the check valve/drain valve assembly 508 to the frame assembly 514 or other element of the cryogenic fluid creation system 400. Further, the check valve/drain valve assembly 508 may include a ball valve 2204 coupled between a first pipe nipple 2206 and a second pipe nipple 2208. The tee 2210 may be coupled to the second pipe nipple 2208.

A first branch of the tee 2210 may extend to a check valve 2212. The check valve 2212 may include any device capable of ensuring that possibly contaminated fluid (e.g., water) does not enter the reservoir assembly 506. A barbed hose adapter 2214 may be coupled to a distal end of the check valve 2212. A second branch of the tee 2210 may be coupled to a third pipe nipple 2216, an elbow 2218, and a barbed hose adapter 2220. The barbed hose adapter 2214 may be coupled to the reservoir assembly 506 via a hose (not shown). Further, the barbed hose adapter 2220 may be coupled to the UVGI assembly 510. The first pipe nipple 2206 may be open to ambient air and pressure such that when the ball valve 2204 is opened, the hoses and other elements of the cryogenic fluid creation system 400 may be drained or bled.

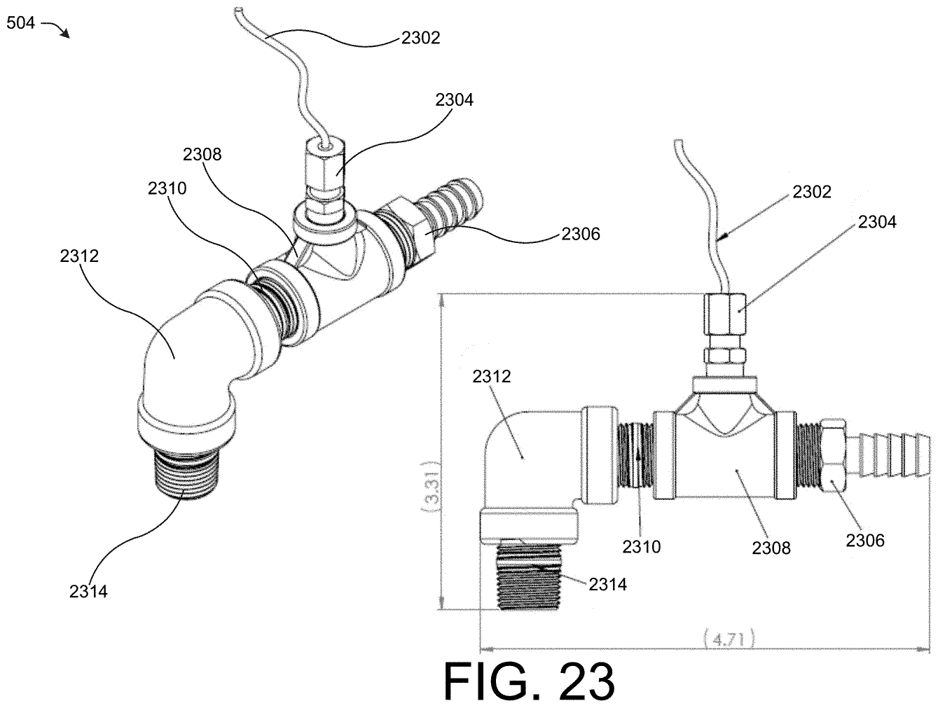

FIG. 23 illustrates a resistance temperature detector (RTD) assembly 504 of a cryogenic fluid creation system 400, according to an example of the principles described herein. The RTD assembly 504 allows for a temperature of a refrigerant used to decrease the internal temperature of the cryogenic fluid generator assembly 502 to be detected. The RTD assembly 504 may be coupled to the shell half coupling 1302-N. A tee 2308 may be coupled to a barbed hose adapter 2306 to receive returned refrigerant that has been circulated within the cryogenic fluid generator assembly 502. In one example, a compressor or other device may be coupled between the shell half coupling 1302-1 and the shell half coupling 1302-N. The tee 2308 may also be coupled to a nipple 2310, an elbow 2312 and nipple 2314. In one example, the nipple 2314 may be coupled to the shell half coupling 1302-N in order to allow the returned refrigerant to enter the cryogenic fluid generator assembly 502.

Further, the tee 2308 may be coupled to a sensor fitting 2304 that houses an RTD sensor. The RTD sensor housed in the sensor fitting 2304 may be coupled to wiring 2302. The wiring 2302 may, in turn, be coupled to the electrical control assembly 402 to allow the electrical control assembly 402 to receive sensor data from the RTD assembly 504.

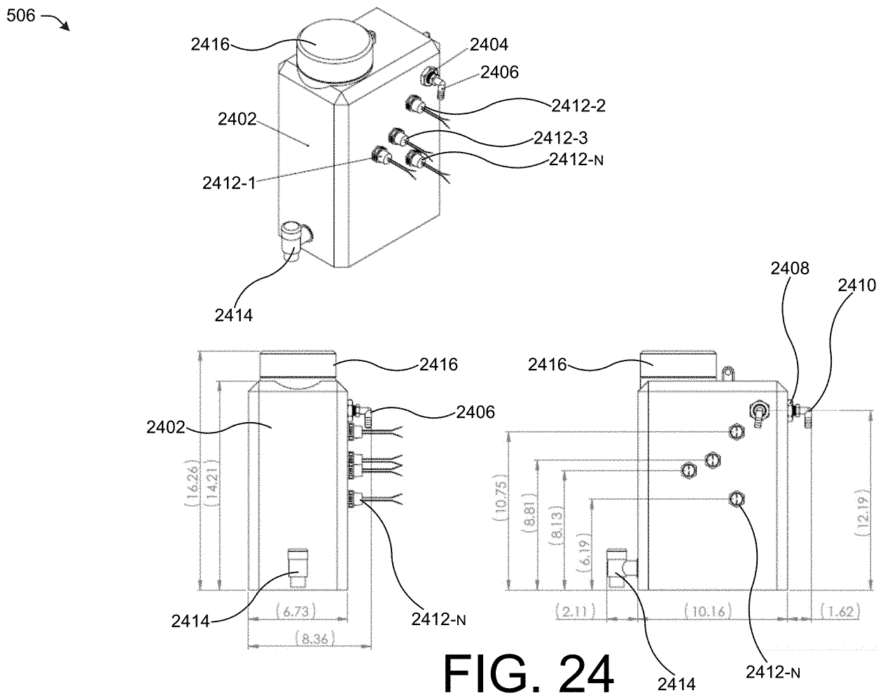

FIG. 24 illustrates a reservoir assembly 506 of a cryogenic fluid creation system 400, according to an example of the principles described herein. The reservoir assembly 506 may include a reservoir 2402 to hold the cryogenic fluid composition. Further, a first through wall adapter 2404 and a first barbed hose elbow 2406 may be coupled to an upper portion of the reservoir 2402. The first barbed hose elbow 2406 may be fluidically coupled to the UVGI assembly 510 so that the filtered and sanitized fluid may enter the reservoir 2402.

The reservoir assembly 506 may further include a second through wall adapter 2408 and a second barbed hose elbow 2410. The second through wall adapter 2408 and a second barbed hose elbow 2410 may be coupled to another fluid source such as water, a cryogenic fluid composition, concentrated cryogenic fluid composition, other chemicals, and combinations thereof to allow for additional components to be added to the reservoir 2402.

The reservoir 2402 may include a number of fluid level probes 2412-1, 2412-2, 2412-3, 2412-N, where N is any integer greater than or equal to 1 (collectively referred to herein as fluid level probe(s) 2412 unless specifically addressed otherwise). The fluid level probes 2412 may include any sensor capable of detecting the presence of fluid within the reservoir 2402. As depicted in FIG. 24, the fluid level probes 2412 may be located at various heights along the reservoir 2402 so that the various levels may be detected. The fluid level probes 2412 may be communicatively and/or electrically coupled to the electrical control assembly 402 so that the electrical control assembly 402 may receive sensor data from the fluid level probes 2412. In one example, the electrical control assembly 402 may display to a user a level of fluid within the reservoir 2402 via, for example, a graphical user interface (GUI) displayed on a display device based on the sensor data obtained from the fluid level probes 2412.

The reservoir 2402 may include a lid 2416. In one example, the lid 2416 may be coupled to the reservoir 2402 via a hinge, a living hinge, a number of mating treads formed on the lid 2416 and reservoir 2402 or other coupling means. The lid 2416 may allow a user to add additional chemicals, for example, to the fluid contained within the reservoir 2402 in preparation for the fluid (e.g., the cryogenic fluid composition) to be fluidically conveyed to the cryogenic fluid generator assembly 502.

The reservoir 2402 may further include an exit port 2414. The exit port 2414 may be fluidically coupled to the cryogenic fluid generator assembly 502 to provide the cryogenic fluid composition to the cryogenic fluid generator assembly 502 for freezing.

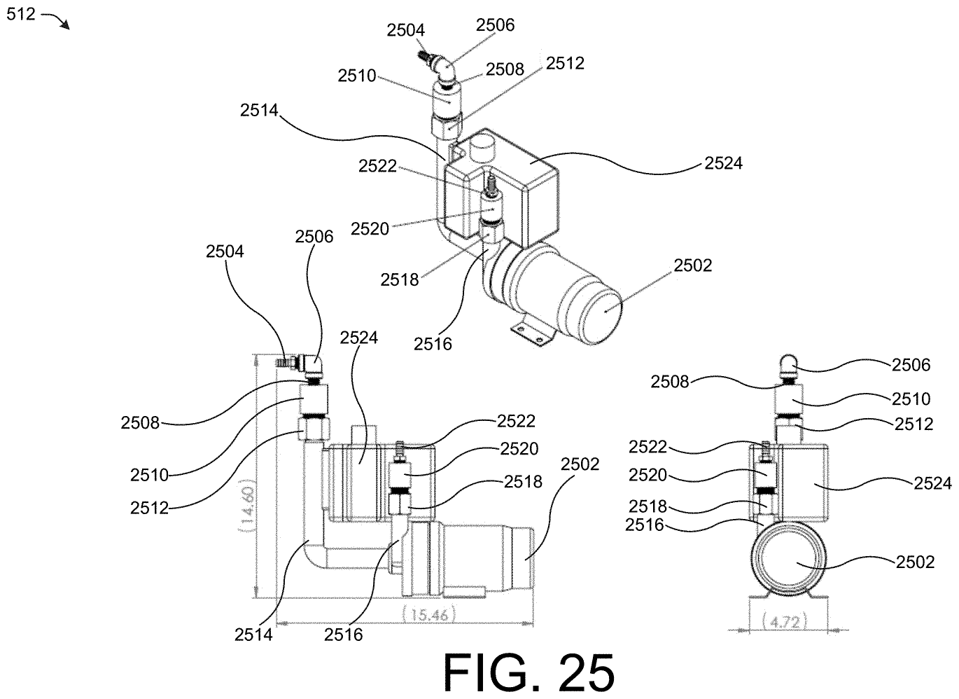

FIG. 25 illustrates a pump 512 of a cryogenic fluid creation system 400, according to an example of the principles described herein. In one example, the pump 512 may include a self-priming pump. In one example, the pump 512 may include a self-priming magnet pump (e.g., for chemical seawater) (Mfg. No. PMDS-421B2M) manufactured and distributed by Sanso Electric. The pump 512 may include a pump 2502. The pump 2502 may include an intake including a barbed hose adapter 2504 coupled to an elbow 2506, a nipple 2508, a threaded pipe fitting 2510, an adapter 2512, and an intake pipe 2514. The pump 2502 may also include an output including an output pipe 2516, an adapter 2518, a threaded pipe fitting 2520, and a barbed hose adapter 2522. Further, in one example, the pump 512 may include a self-priming tank 2524.

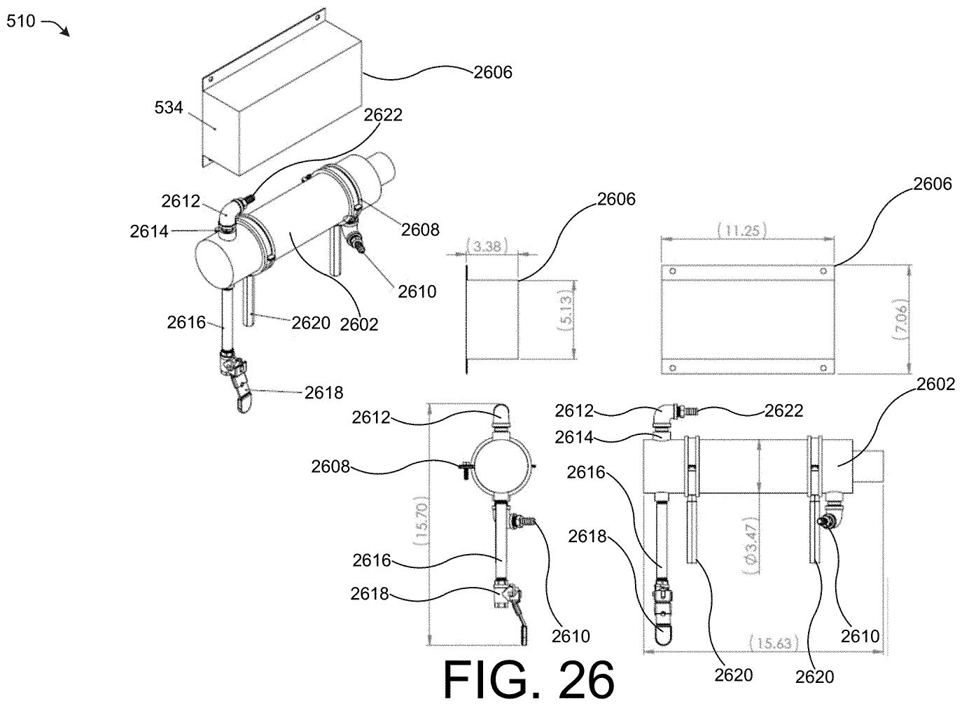

FIG. 26 illustrates an ultraviolet germicidal irradiation (UVGI) assembly 510 of a cryogenic fluid creation system 400, according to an example of the principles described herein. In one example, the UVGI assembly 510 may include any ultraviolet-c (UVC) light-emitting diode (LED) sterilizer assembly that provides anti-germicidal radiation to sterilize any fluid introduced therein including water, cryogenic fluid compositions, and other fluids. The UVGI assembly 510 may include a UVGI sanitization unit 2602 and a power source 534. The power source 534 may include a power source housing 2606 and may include any source of electrical power to cause a UVC LED within the UVGI sanitization unit 2602 to illuminate. Thus, the power source 534 may be electrically coupled to the UVC LED within the UVGI sanitization unit 2602 via a number of electrical wires (not shown). The power source 534 may be controlled and/or receive electrical power from the electrical control assembly 402. The source of electrical power for the cryogenic fluid creation system 400 and all of its various elements may be provided from a mains alternating current/direct current (AC/DC) power source such as the electrical power provided to a facility through an electrical power grid. The power source housing 2606 may be secured to the frame assembly 514 and secure the power source 534 to the cryogenic fluid creation system 400.

The UVGI sanitization unit 2602 may include a number of clamping hangers 2608 used to couple the UVGI sanitization unit 2602 to the frame assembly 514. Further, the UVGI sanitization unit 2602 may include a number of hexagonal standoff posts 2620 to ensure that the UVGI sanitization unit 2602 is positioned within the frame assembly 514 away from a number of other elements including, for example, the pump 512.

The UVGI sanitization unit 2602 may be fluidically coupled to the pump 512 via a barbed hose adapter 2610 coupled to the UVGI sanitization unit 2602. The barbed hose adapter 2610 may be coupled to the pump 512 via a hose (not shown). Further, the UVGI sanitization unit 2602 may be fluidically coupled to the reservoir assembly 506 via a nipple coupled to the UVGI sanitization unit 2602 and an elbow 2612, a nipple 2614, and barbed hose adapter 2622. The barbed hose adapter 2622 may be fluidically coupled to the reservoir assembly 506 via a hose (not shown).

The UVGI sanitization unit 2602 may further include a nipple 2616 coupled to the UVGI sanitization unit 2602 and a ball valve 2618 may be fluidically coupled to the nipple 2616. The ball valve 2618 may be used to drain the UVGI sanitization unit 2602 after use so that any fluid (e.g., water, cryogenic fluid compositions, etc.) within the UVGI sanitization unit 2602 may not become contaminated through remaining stagnant.

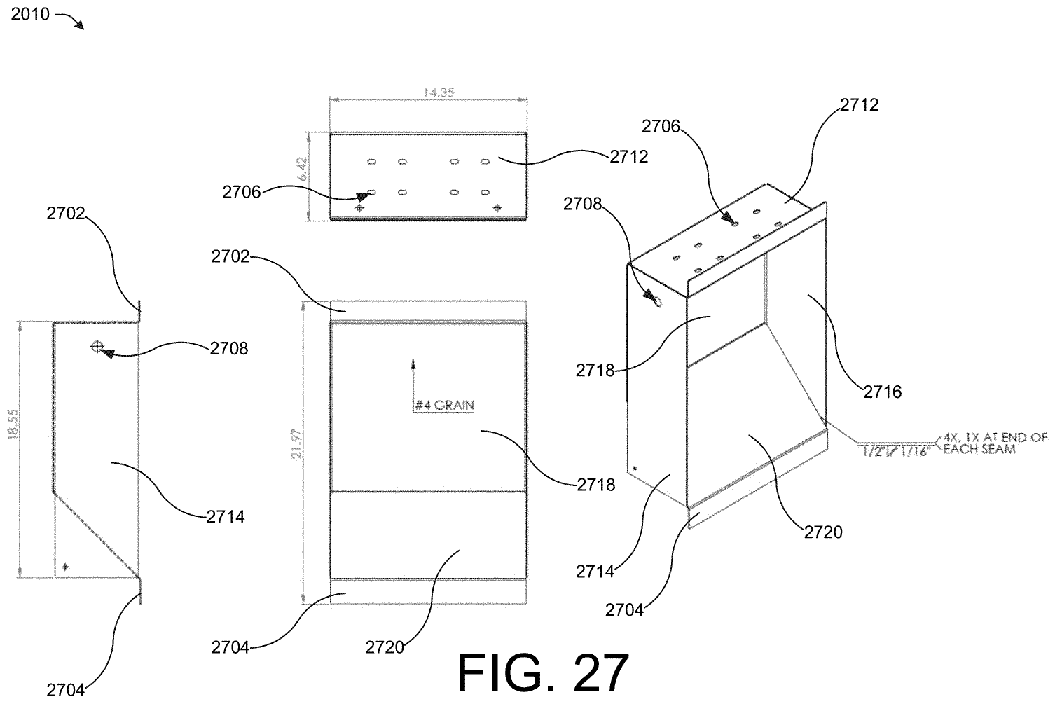

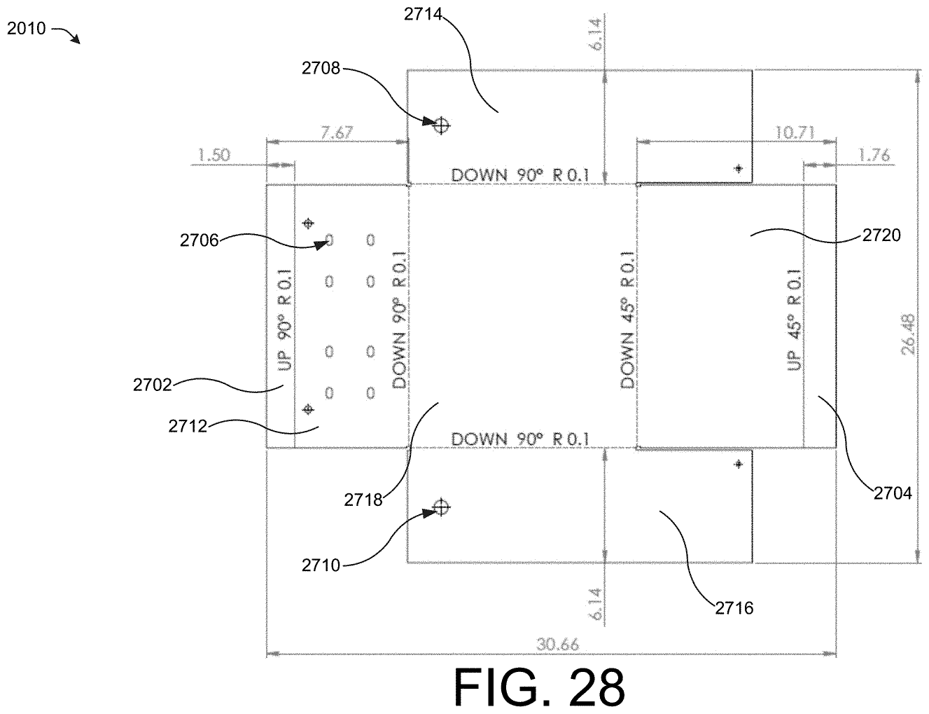

FIGS. 27 and 28 illustrates a water filter housing 2010 of a cryogenic fluid creation system, according to an example of the principles described herein. The water filter housing 2010 may include a first interface 2702 and a second interface 2704 to couple the water filter housing 2010 to, for example, the frame assembly 514 so that the water filter housing 2010 may support the fluid filter devices 2002. A number of fastener apertures 2706 may be defined in a top 2712 of the water filter housing 2010 to allow for fasteners to be extended therethrough and couple the water filter housing 2010 to, for example, the frame assembly 514.

The water filter housing 2010 may further include a first side 2714 and a second side 2716. Further, the water filter housing 2010 may include a vertical back portion 2718 and a slanted back portion 2720. A first side aperture 2708 may be defined in the first side 2714 to accommodate for the inlet port 2004 entering the water filter housing 2010 and coupling to the fluid filter devices 2002. A second side aperture 2710 may be defined in the second side 2716 to accommodate for the outlet port 2008 coupling to the fluid filter devices 2002 and exiting the water filter housing 2010.

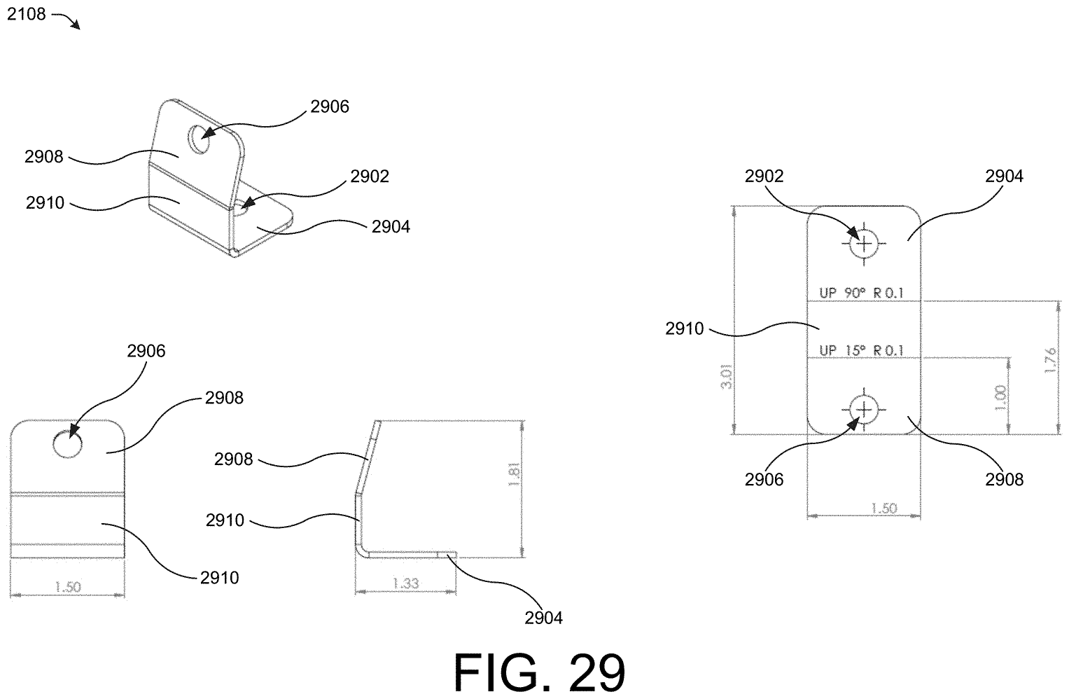

FIG. 29 illustrates electrical box bracket 2108 of a cryogenic fluid creation system 400, according to an example of the principles described herein. The electrical box bracket 2108 is depicted in FIG. 21 in association with the frame assembly 514 and is referred to therein as a “bracket 2108”. The electrical box bracket 2108 may be used to couple any element of the cryogenic fluid creation system 400 and is described herein as coupling the electrical control assembly 402 to the frame assembly 514 as an example. The electrical box bracket 2108 may include a first aperture 2902 defined in a base portion 2904. A fastener may be extended through the first aperture 2902 and engage with one of the slotted frame pieces 2102 of the frame assembly 514. Further, the electrical box bracket 2108 may include a second aperture 2906 defined in an angled top portion 2908. A fastener may be extended through the second aperture 2906 and engage with one of the elements of the cryogenic fluid creation system 400 such as, for example, the electrical control assembly 402. The electrical box bracket 2108 has a general L-shaped cross section that includes the angled top portion 2908 angled at approximately a 15° with respect to a bottom portion 2910 with the angled top portion 2908 and the bottom portion 2910 forming the vertical portion of the L-shaped cross section. Further, the base portion 2904 may be coupled to the bottom portion 2910 at approximately a 90° angle with respect to the bottom portion 2910.

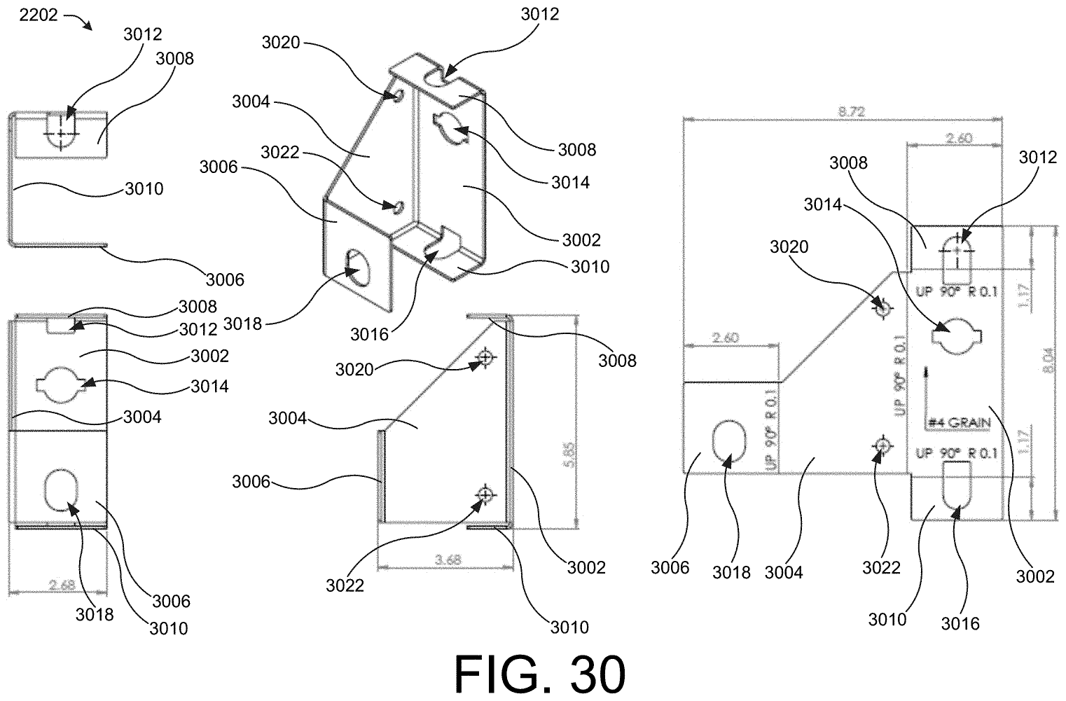

FIG. 30 illustrates a valve bracket 2202 of a cryogenic fluid creation system 400, according to an example of the principles described herein. The valve bracket 2202 may be used to couple the check valve/drain valve assembly 508 to, for example, the frame assembly 514 or other element of the cryogenic fluid creation system 400. The valve bracket 2202 may include a back portion 3002, a side portion 3004 formed at a 90° angle with respect to the back portion 3002 about a vertical axis, and a front portion 3006 formed at a 90° angle with respect to the side portion 3004 about a vertical axis. Further, a top portion 3008 may be formed at a 90° angle with respect to the back portion 3002 about a horizontal axis. A bottom portion 3010 may be formed at a 90° angle with respect to the back portion 3002 about a horizontal axis.

A first aperture 3012 may be defined in the top portion 3008 and back portion 3002 along a transition between the top portion 3008 and back portion 3002. The first aperture 3012 may be formed to allow the first pipe nipple 2206 to extend out of the valve bracket 2202 and support the first pipe nipple 2206 within the valve bracket 2202. The valve bracket 2202 may further include a second aperture 3014 defined in the back portion 3002. The second aperture 3014 may be formed to allow the ball valve 2204 to extend out of the valve bracket 2202 and support the ball valve 2204 within the valve bracket 2202. The valve bracket 2202 may further include a third aperture 3016 defined in the back portion 3002 and the bottom portion 3010 along a transition between the back portion 3002 and the bottom portion 3010. The third aperture 3016 may be formed to allow the tee 2210 and the third pipe nipple 2216 to extend out of the valve bracket 2202 and support the tee 2210 and the third pipe nipple 2216 within the valve bracket 2202. A fourth aperture 3018 may be defined in the front portion 3006. The fourth aperture 3018 may be formed to allow the check valve 2212 to extend out of the valve bracket 2202 and support the check valve 2212 within the valve bracket 2202. Further, a first coupling aperture 3020 and a second coupling aperture 3022 may be defined in the side portion 3004 or elsewhere on the valve bracket 2202 to allow the valve bracket 2202 to be coupled to the frame assembly 514 or other elements of the cryogenic fluid creation system 400.

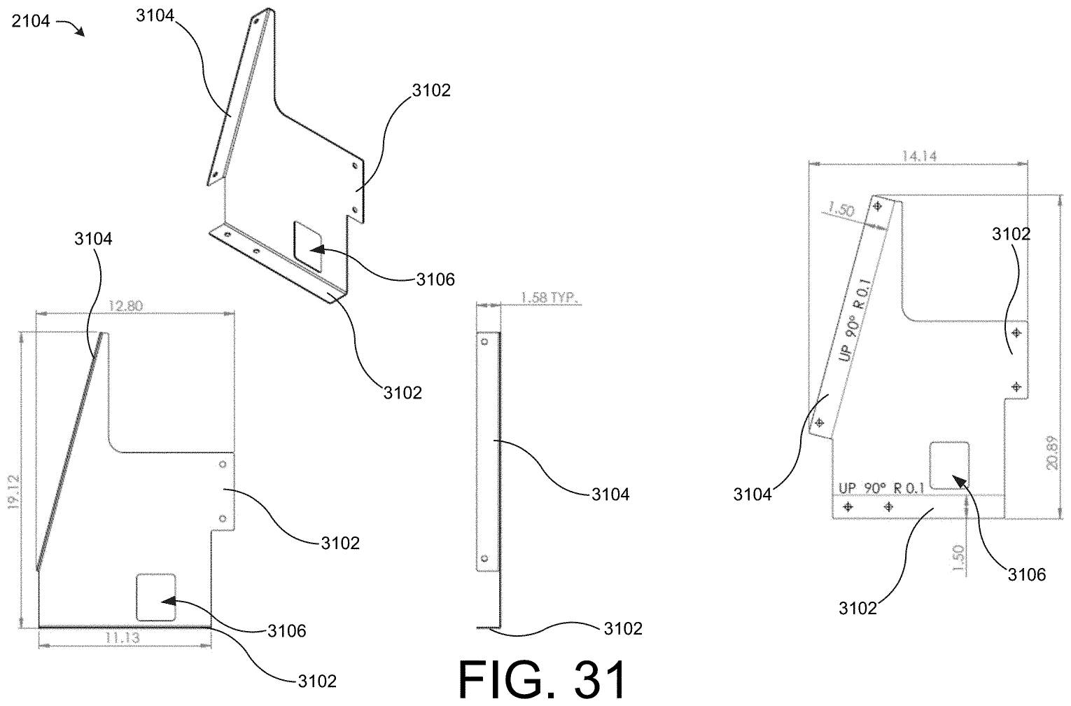

FIG. 31 illustrates an electrical box mount 2104 of a cryogenic fluid creation system 400, according to an example of the principles described herein. In one example, the electrical box mount 2104 may be used to mount the electrical control assembly 402 to the cryogenic fluid creation system 400. The electrical box mount 2104 may include a number of mounting points 3102 to allow the electrical box mount 2104 to be coupled to the frame assembly 514 such as slotted frame pieces 2102-1 and 2102-5. The electrical box mount 2104 may further include a device mounting point 3104. The device mounting point may be used to mount, for example, the electrical control assembly 402 to the frame assembly 514. A cable aperture 3106 may be defined in the electrical box mount 2104 to allow for electrical wiring and cables to be fed from the electrical control assembly 402 to other elements within the cryogenic fluid creation system 400 and to assist in managing the routing of the electrical wiring and cables.

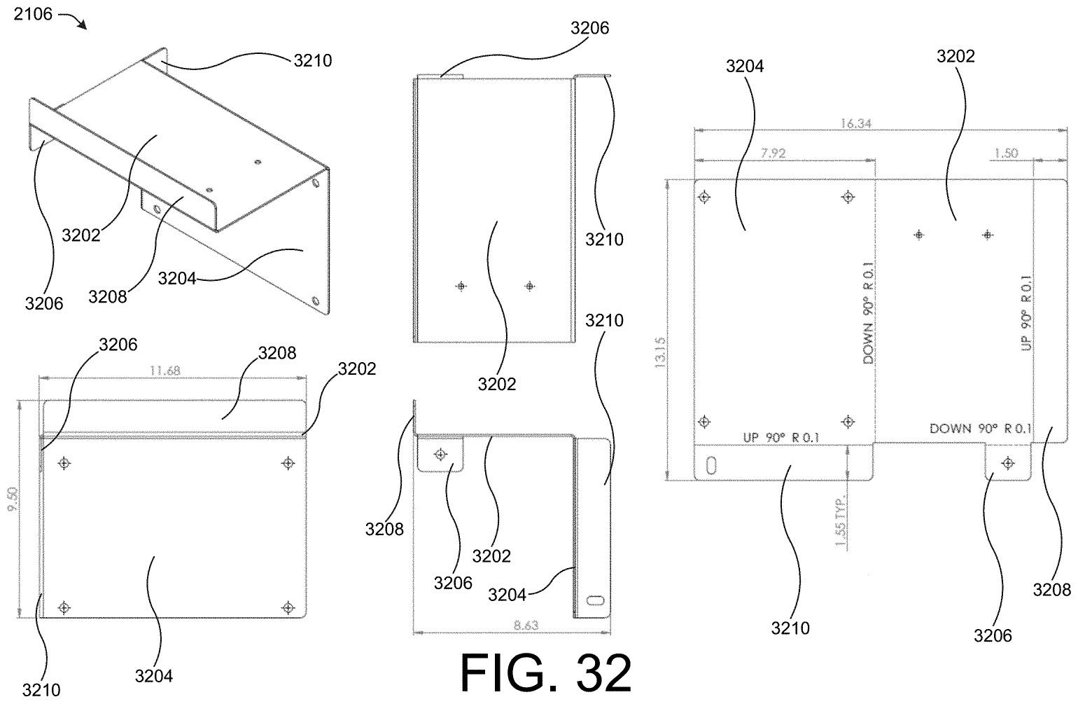

FIG. 32 illustrates a reservoir shelf 2106 and sanitizer mount of a cryogenic fluid creation system 400, according to an example of the principles described herein. The reservoir shelf 2106 may serve to support the reservoir assembly 506 on the shelf portion 3202 and/or couple the reservoir assembly 506 to, for example, the shelf portion 3202. Further, the reservoir shelf 2106 may include a first tab 3206 and a second tab 3210 that may be used to couple the reservoir shelf 2106 to, for example, the slotted frame pieces 2102-1 and 2102-5 of the frame assembly 514. In this manner, the reservoir shelf 2106 may be coupled to the frame assembly 514 so that the reservoir shelf 2106 may support other elements of the cryogenic fluid creation system 400. The reservoir shelf 2106 may further include a vertical portion 3204. The vertical portion 3204 may be used to couple the power source 534 of the UVGI assembly 510 to the reservoir shelf 2106 and support the power source 534 within the cryogenic fluid creation system 400. The shelf portion 3202 may include a shelf end 3208 to secure the reservoir assembly 506.

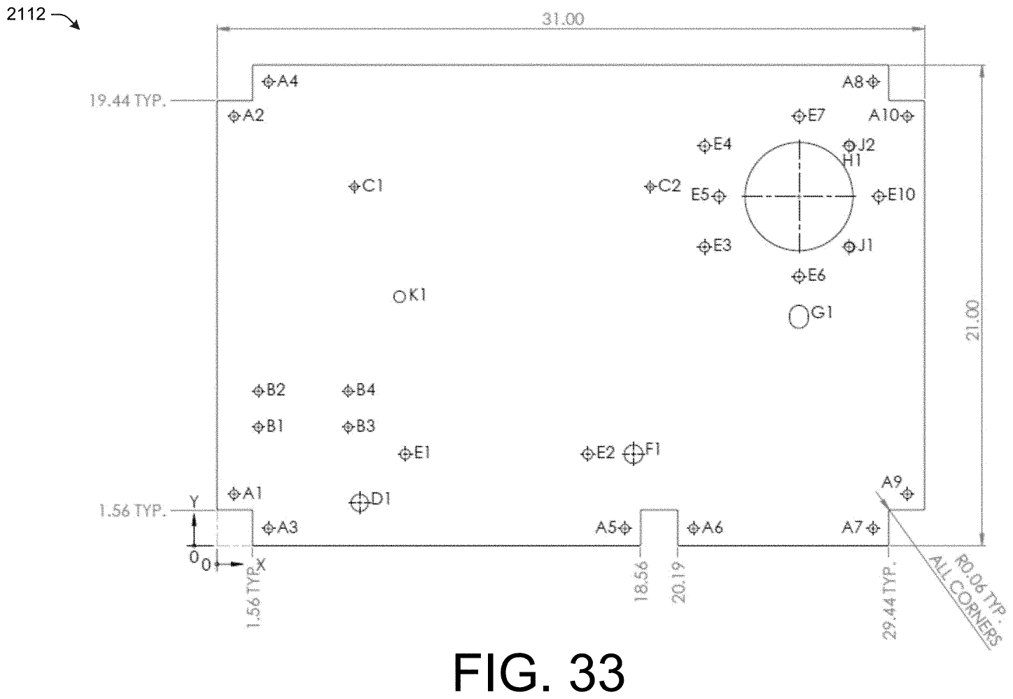

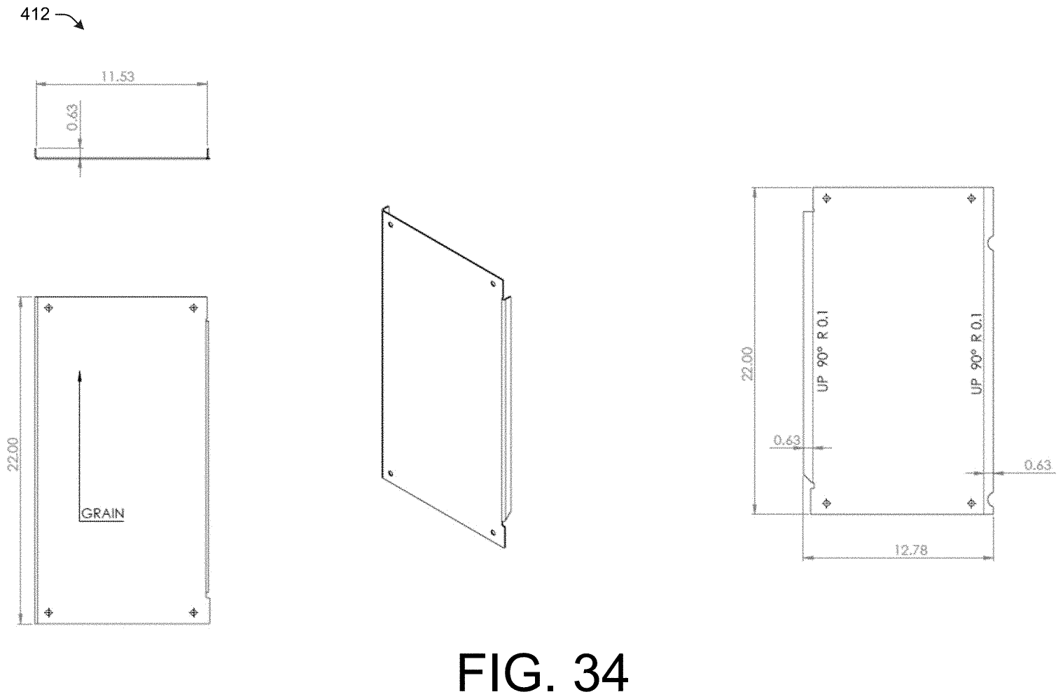

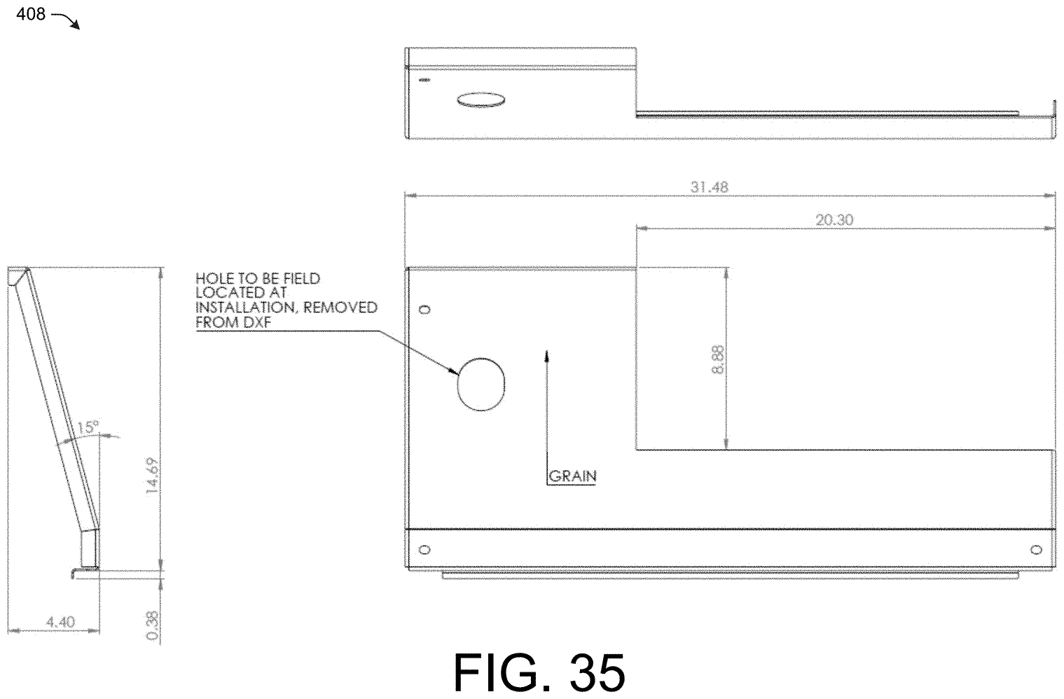

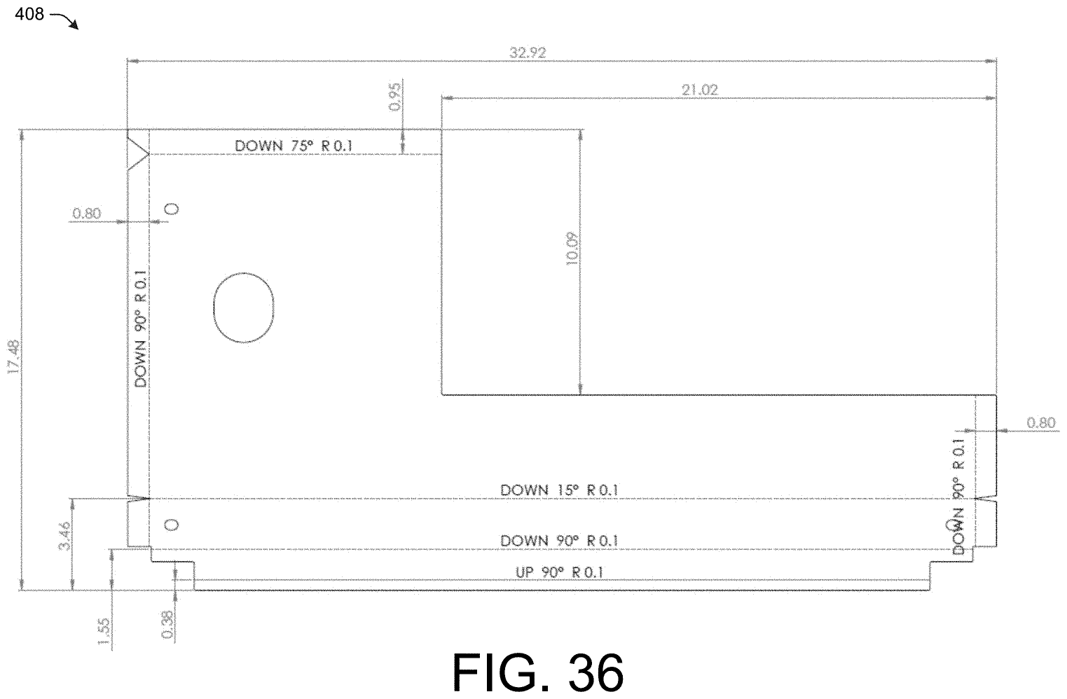

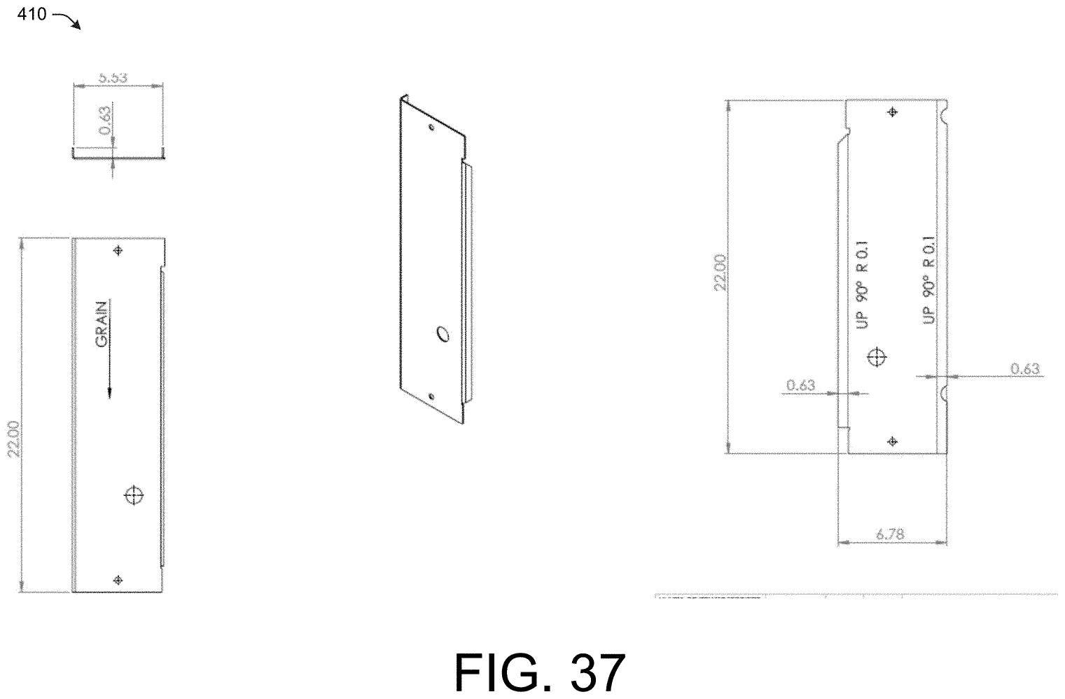

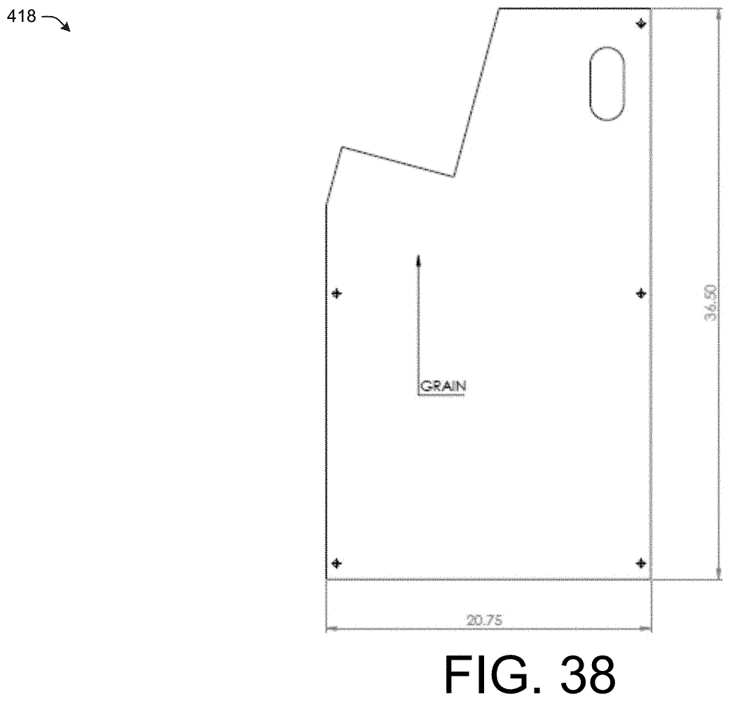

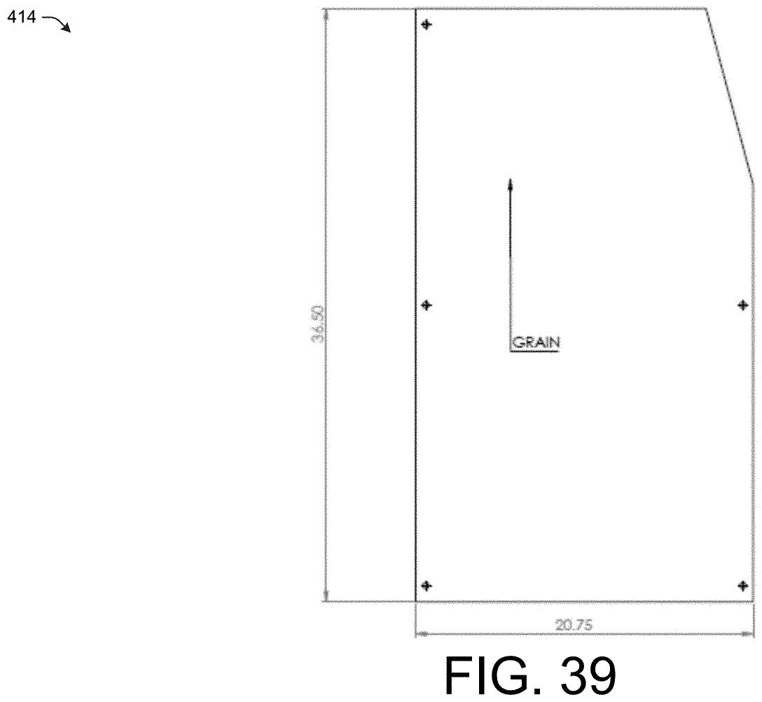

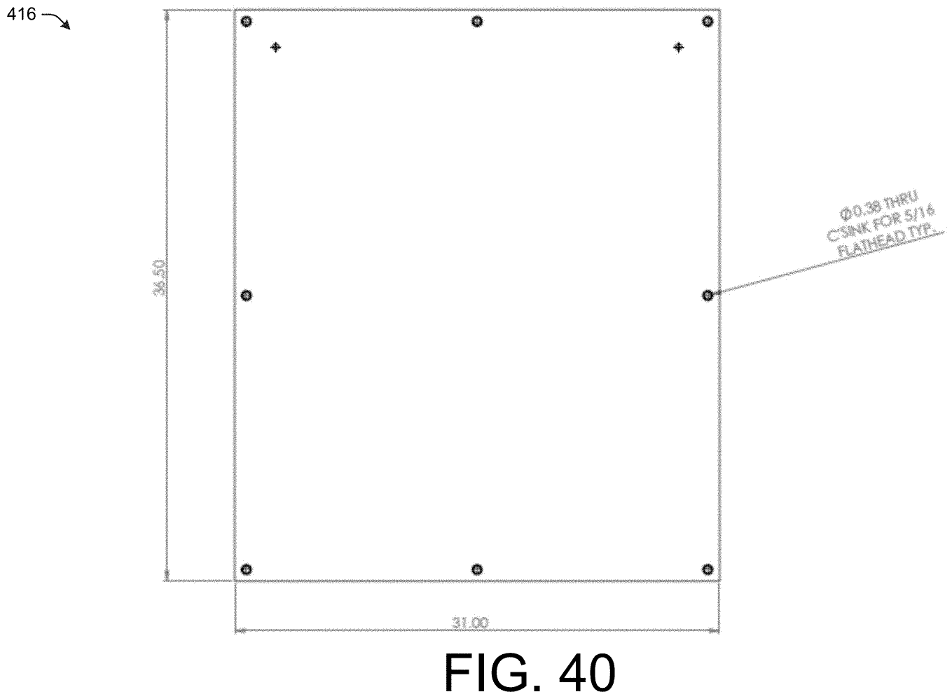

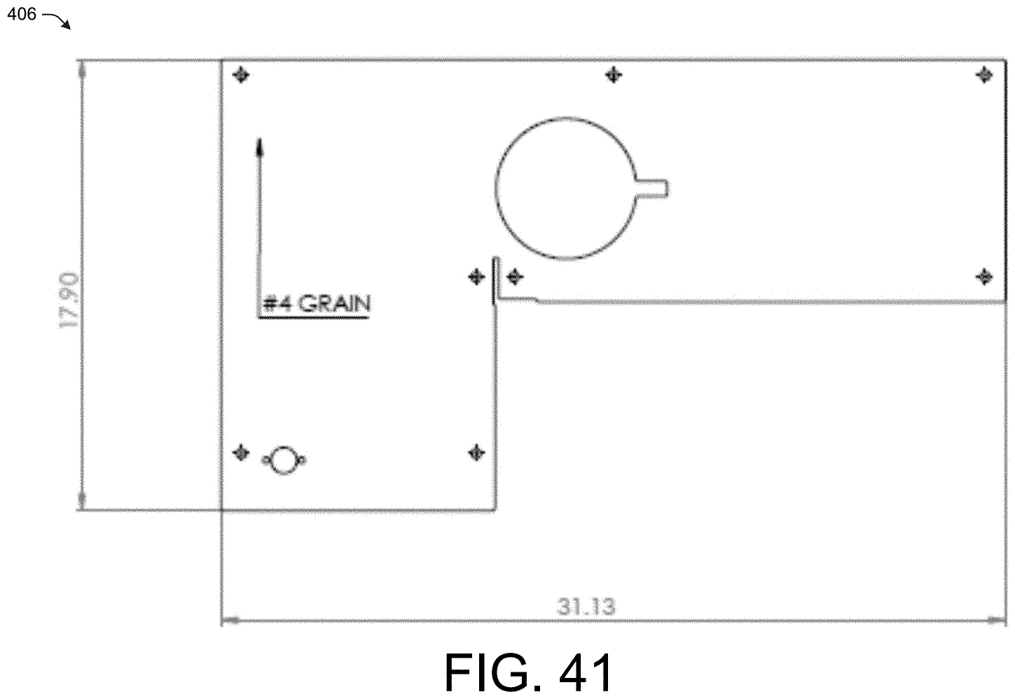

As described above, a number of housing guards or plates may be coupled to the frame assembly 514 in order for the elements within the cryogenic fluid creation system 400 to be covered, secured, and protected from exterior influences so that the cryogenic fluid creation system 400 may function as intended. FIG. 33 illustrates a base plate 2112 of a cryogenic fluid creation system 400, according to an example of the principles described herein. FIG. 34 illustrates a front lower left housing guard 412 of a cryogenic fluid creation system 400, according to an example of the principles described herein. FIGS. 35 and 36 illustrate a front, upper housing guard 408 of a cryogenic fluid creation system 400, according to an example of the principles described herein. FIG. 37 illustrates a front lower right housing guard 410 of a cryogenic fluid creation system 400, according to an example of the principles described herein. FIG. 38 illustrates a right side housing guard 418 of a cryogenic fluid creation system 400, according to an example of the principles described herein. FIG. 39 illustrates a left side housing guard 414 of a cryogenic fluid creation system 400, according to an example of the principles described herein. FIG. 40 illustrates a rear housing guard 416 of a cryogenic fluid creation system 400, according to an example of the principles described herein. FIG. 41 illustrates a top housing guard 406 of a cryogenic fluid creation system 400, according to an example of the principles described herein.

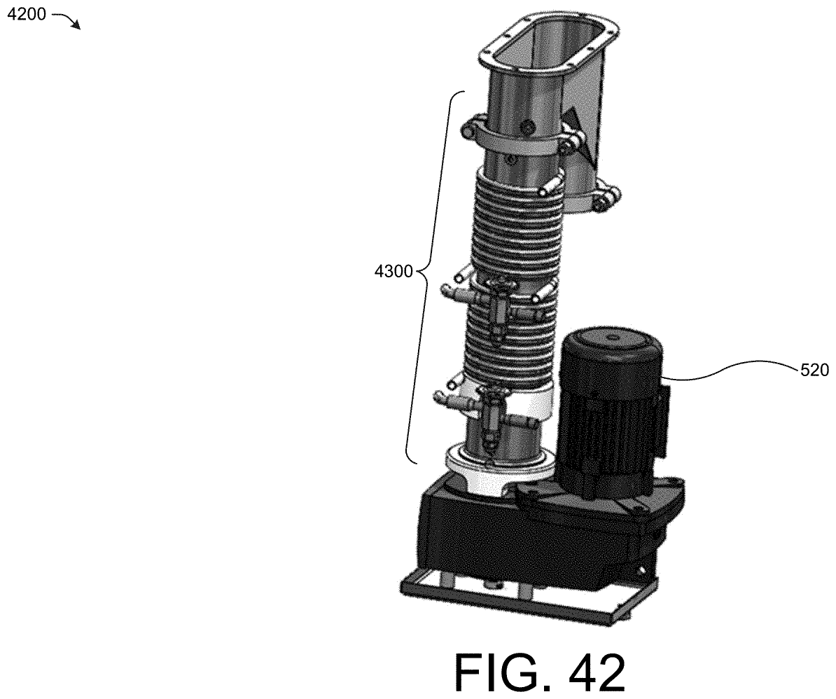

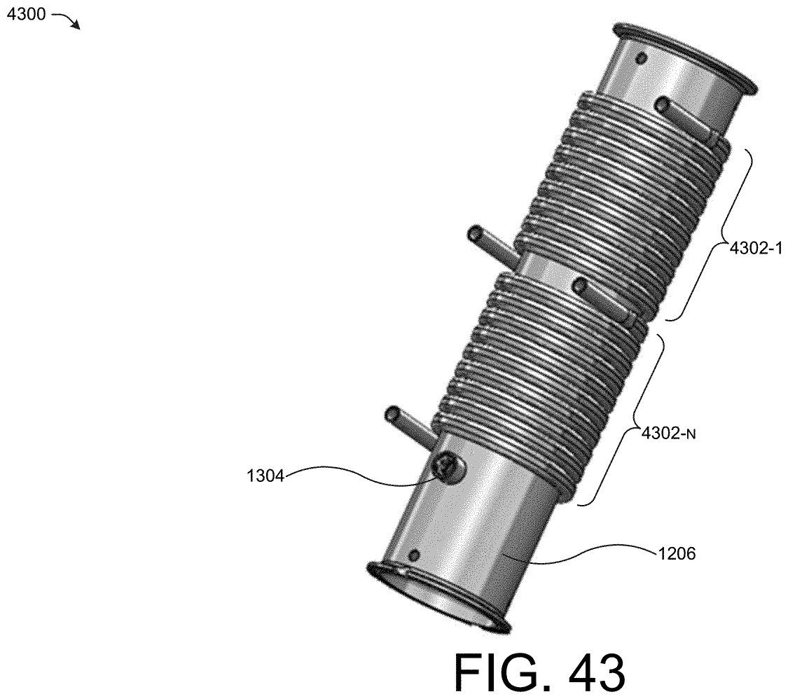

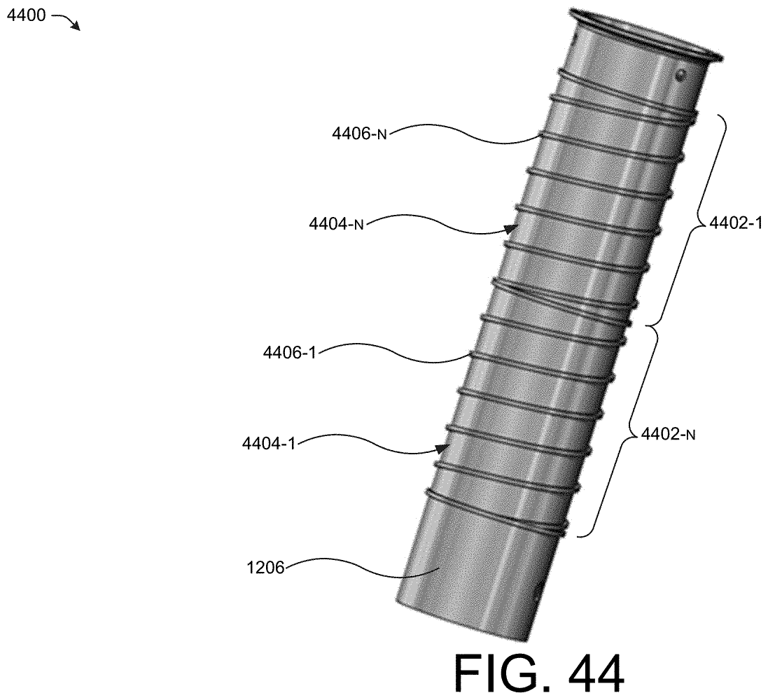

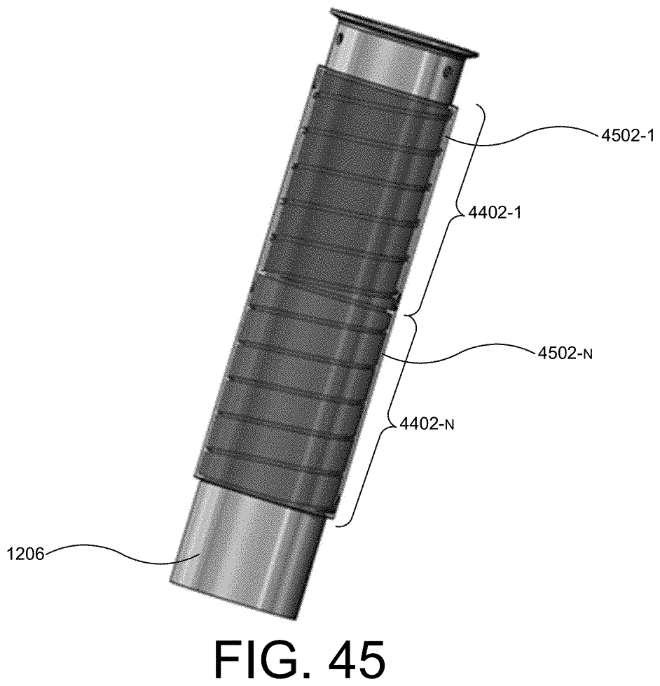

FIG. 42 illustrates a heat exchanger system 4200 of a cryogenic fluid creation system 400, according to an example of the principles described herein. FIG. 43 illustrates a heat exchanger 4300 of a cryogenic fluid creation system 400, according to an example of the principles described herein. FIG. 44 illustrates an internal chamber 4400 of a heat exchanger of a cryogenic fluid creation system 400, according to an example of the principles described herein. FIG. 45 illustrates a number of intermediate sleeves 4502 of an internal chamber 4400 of a heat exchanger 4200 of a cryogenic fluid creation system 400, according to an example of the principles described herein. The example of the heat exchanger system 4200 of FIGS. 42-45 may include a motor 520 mechanically coupled to an auger 800 (not shown) within a heat exchanger 4300.

The heat exchanger 4300 may include a plurality of internal helical coils 4302-1, . . . 4302-N, where N is any integer greater than or equal to 1 (collectively referred to herein as internal helical coil(s) 4302 unless specifically addressed otherwise) surrounding a core 1206. In the example of FIG. 43, the auger 800 resides within the core 1206 of the heat exchanger 1202. In one example, refrigerant tubing such as, for example, copper tubing, may be wrapped around the outside of the core 1206 whereby refrigerant is circulated through the refrigerant tubing. The double chamber arrangement of two inlets and two outlets greatly increases the freezing efficiency and molecular nanoparticles (e.g., nano-ice) formation. The refrigerant tubing may be used whereby the cooling transfer passes through the walls of the refrigerant tubing. Although functional, the inefficient contact surface to the core 1206 of the heat exchanger 1202 (e.g., a stainless steel cylinder) may become corroded decreasing the efficiency of the transfer of heat. Further, the heat exchange must also permeate through the wall of the core 1206 of the heat exchanger 1202 (e.g., a stainless steel cylinder) in order to freeze the cryogenic fluid composition.

In contrast, the example of FIG. 44 provides a design that greatly enhances cooling transfer. The example of FIG. 44 includes a number of spiral chambers 4404-1, . . . 4404-N, where N is any integer greater than or equal to 1 (collectively referred to herein as spiral chamber(s) 4404 unless specifically addressed otherwise). The spiral chambers 4404 may be formed by the formation of a number of spiral bars 4406-1, . . . 4406-N, where N is any integer greater than or equal to 1 (collectively referred to herein as spiral bar(s) 4406 unless specifically addressed otherwise). In one example, the spiral bars 4406 may be monolithically formed on the core 1206 of the heat exchanger 1202. Further, in one example, the spiral bars 4406 may be oriented in a spiral manner around the circumference of the core 1206 such that the spiral chambers 4404 are also oriented in a spiral manner around the circumference of the core 1206. The intermediate sleeves 4502 depicted in FIG. 45, when engaged around the upper outer chamber 4402-1 and a lower outer chamber 4402-N and their respective spiral chambers 4404 and spiral bars 4406 serve to contain the refrigerant within the spiral chambers 4404 and between the spiral bars 4406. In this manner, refrigerant introduced into the spiral chambers 4404 may directly interface with the core 1206 of the heat exchanger 1202 rather than indirectly as depicted in the example of FIG. 43. Further, in the arrangement depicted in FIGS. 44 and 45, the refrigerant may still be circulated into two separate chambers in a similar manner as depicted in the example of FIG. 43, but the heat transfer provided in the example of FIGS. 44 and 45 only has to occur between the stainless steel wall of the core 1206.

The heat exchanger 4300 of FIGS. 42-45 benefits from a “dual chamber” with the internal helical coils 4302 that direct a refrigerant upward in a circulatory and/or rotary manner at the same time in an upper outer chamber 4402-1 and a lower outer chamber 4402-N, where N is any integer greater than or equal to 1 (collectively referred to herein as outer chamber(s) 4402 unless specifically addressed otherwise) defined by the positions of the internal helical coils 4302. The internal helical coils 4302 may be produced by coiling tubes or square or round solid stock material to create helical channels spacings. In this manner, the upper and lower outer chambers 4402 allow for one of the outer chambers 4402 to be cooled at a different degree relative to the other outer chamber 4402 based on how the cryogenic fluid composition is to be frozen into the nano-ice structure cryogenic fluid. A number of intermediate sleeves 4502-1, . . . 4502-N, where N is any integer greater than or equal to 1 (collectively referred to herein as intermediate sleeve(s) 4502 unless specifically addressed otherwise) of the internal chamber 4400 may be include for as many outer chambers 4402 as are included in the heat exchanger 4300.

The preparation of the cryogenic fluid by freezing the cryogenic fluid composition into a nano-ice slurry may be brought about by including any formulation of SERAKUL cryogenic fluid developed, manufactured, and/or distributed by Glacia, Inc. Applications of the cryogenic fluid depicted and described herein may include, for example, those listed in Table 1. The formulations of the cryogenic fluid composition may include those described herein in Table 2. Table 3 describes a number of applications of the methods, systems, devices, and formulations for the cryogenic fluid described herein. However, the lists of information provided in Tables 1 through 3 are not exhaustive.

TABLE 1
Applications of the cryogenic fluid

| | | |
|---|---|---|
| Direct application | Athletic (human, equine, other), | Wet applications— |
| on or | medical (human, equine, other), | direct contact when |
| submersion in. | other | appliedtoor |
| | | submergedwithin |
| | | boots, buckets, bowls, |
| | | bags and/or any type of |
| | | other vessel which |
| | | holds or contains the |
| | | product and/or is |
| | | applied directly upon a |
| | | body part/limb/digit, |
| | | etc. |
| Wraps or bags— | Athletic (human, equine, other), | Dryorwet |
| various forms | medical (human, equine, other), | applications |
| and materials | other | depending upon type |
| whether | | of wrap and material. |
| custom | | |
| and/or OEM | | |
| Other | Athletic (human, equine, other), | Dry or wet in any and |
| | medical (human, equine, other), | all forms whatsoever. |
| | other | |
| Tubes, needles, | Athletic, medical, consumer, | Product contained |
| hoses, pipes, | electronic, manufacturing and/or | therein. |
| etc. | any other | |

### The Applications of the Cryogenic Fluid

Formulations of the cryogenic fluid may include, for example, those listed in Table 2.

TABLE 2
Formulations

| Ingredients | Percentage |
|---|---|
| Sodium Chloride—NaCL | 3% |
| Sodium Chloride—NaCL | 3% and 1% |
| and Magnesium sulfate, | |
| MgSO4 | |
| Sodium Chloride—NaCL | 3% |
| Sodium Chloride—NaCL | 3% and 1% |
| and Magnesium sulfate, | |
| MgSO4 | |

The cryogenic fluid composition may include water (H₂0), and at least one salt. The ratio of water to the at least one salt is approximately between 1% and 6% salt with the remainder water. The ratio may be measured by weight. The ratio may be measured by volume. The cryogenic fluid may be formed between 20° F. and 31° F. The shape of ice particles within the cryogenic fluid may include at least one of approximately round, oblong, or globular, and may include a roughness average (RA) of between 63 RA and 125 RA. The diameter of ice particles within the cryogenic fluid may be between 1 nanometer and 900 micrometers. The at least one salt may include Sodium Chloride (NaCl) and magnesium sulfate (MgSO₄). The cryogenic fluid may further include at least one of an alcohol, a sugar, the at least one salt, and combinations thereof. The cryogenic fluid may further include at least one therapeutic.

The field of use of the methods, systems, devices, and formulations for the cryogenic fluids may include, for example, those listed in Table 3.

TABLE 3
Applications of methods, systems, devices, and formulations for the cryogenic fluids

| | Application/Idea/ | |
|---|---|---|
| | Theory | Description |
| Veterinary— | | |
| Equine | | |
| | Equine Laminitis & Leg | Penetration through the hoof and deep into the leg |
| | Health | provides competitive advantage over current |
| | | practice |
| | Equine Stifle Joint | Able to penetrate deep into horse leg and cool |
| | Cooling | stifle joint. |
| | Equine Cooling Blanket | Post Race/Competition Cool Down and |
| | | Rehabilitation |
| | Animal Surgery | Product can be delivered via small hose and cool |
| | localizedcooling, | precise areas via sterilized 28 degree flow. |
| | internal & external | |
| Veterinary— | | |
| General | | |
| | Animal Inflammation | Product can be delivered via small hose and cool |
| | Control—injury and | precise areas via sterilized 28 degree flow or more |
| | surgery | broadly applied either directly or with wraps or |
| | | vessels. |
| | Organic Animal Pain | Post procedure, prior to wake up, inflammation |
| | Relief | reduction and pain control. |
| Athletic | | |
| Equine | | |
| | Equine Cool Down and | Post Race/Competition Cool Down |
| | Post Workout Treatment | |
| | Equine Cooling Blanket | Post Race/Competition Cool Down |
| | Equine Daily Problem | Post Race/Competition Cool Down |
| | Area and General Health | |
| | Treatments | |
| Athletic | | |
| Human | | |
| | Human Athletic | Muscle and soft tissue cooling with deep soothing |
| | Pre/During/Post workout | penetration |
| | or competition or event. | |
| | Human Athletic | Body or limb contusion and/or head concussion |
| | Trauma Point of Injury | treatment, rehabilitation and recovery. |
| | treatment | |
| | Human Athletic—Post | Site of surgery inflammation control |
| | Surgery Inflammation | |
| | Human Athletic | Pre competition/intra competition internal cool |
| | Consumable | down |
| Medical | | |
| Surgery | | |
| | Human Medical | Targeted or no-targeted cooling of specific joints/ |
| | Orthopedicsurgery | tissues in/ex situ |
| | targeted cool down | |
| | Human Medical—Site | Targeted or non-targeted cooling of specific |
| | Specific Cooling | joints/tissues in/ex situ |
| | Human Medical - Post | Targeted or non-targeted cooling of specific |
| | Surgery Inflammation | joints/tissues in/ex situ |
| | Control | |
| | Human Medical—Post | Targeted or non-targeted cooling of specific |
| | Surgery Wound Care? | joints/tissues in/ex situ |
| | Human Medical—Plastic | During and/or Post treatment and recovery |
| | Surgery Recovery | |
| Medical | | |
| Organ | | |
| Transplant | | |
| | Human Medical—Organ | Pre-extraction, in situ cool down |
| | cooling in situ | |
| | Human Medical—Organ | Post-extraction, ex situ temperature control and |
| | cooling ex situ | preservation |
| | Human Medical—Organ | Ex Situ, transplantable organs can require cooling |
| | cooling - pre transport | for 2-18 hours. |
| | Human Medical—Organ | Transportation care and temperature control |
| | temp control during | |
| | transport | |
| | Human Medical—Organ | Long term storage, shelflife extension. |
| | storage pre-procedure | |
| Medical | | |
| Ortho/Joint | | |
| | Arthritis Inflammation | Single and/or regular/repeated treatments for |
| | Treatment | inflammation/arthritis comfort and quality of life |
| | Rheumatoid Arthritis | Single and/or regular/repeated treatments for |
| | Treatment | inflammation/arthritis comfort and quality of life |
| | Fracture/Sprain—rapid | Immediate and after-injury treatment and |
| | anti-inflammatory | rehabilitation. |
| Medical | | |
| Other | | |
| | Human Medical—Fever | Bath and/or blanket, wet or dry fever cool-down. |
| | Cool Down | |
| | Human Medical | Treatment during chemotherapy to comfort and |
| | Chemo Cryo Cap | control hair loss. |
| | Human Medical—Bum | Product made with formula consisting of pure |
| | and Sunbum Treatment | saline and/or aloe vera and/or other for treatment |
| | | and rehabilitation. |
| | Human Medical | Slow down metabolism, usually used as a |
| | Protective Hypothermia | lifesaving procedure. |
| | Ambulatory/ | Various uses for ambulatory Product |
| | AmbulanceMounted | |
| | cooling | |
| | Cadaver Preservation— | Tissue can be preserved for weeks, without smell |
| | | decay, etc. |
| | Vaccine Transportation | Temperature controlled transport and storage. |
| | and Storage | |
| Medical | | |
| Research | | |
| | Sample Preservation and | Preservation of original tissue, bone, cell samples |
| | Transportation | in as close to original form as possible. |
| | Tissue and cadaver | Preservation of research samples - extends |
| | preservation | viability of samples. |
| Power/ | | |
| Energy | | |
| Related | | |
| | Thermal Energy Storage | Thermal storage system for off-peak cooling |
| | Systems | needs |
| | Server Rack Cooling | Direct cooling of computer and/or other server |
| | | rack components through closed loop system |
| | Hydronic Cooling in any | Closed Loop cooling with glycol or suitable |
| | and all applications | substitute |
| | IndustrialProcess | Variability in ice making process water allows for |
| | cooling | use of chemicals used in industrial processes in |
| | | the ice-making process itself |
| | Nuclear Reactor Cooling | Can be made with filtered ocean water—potential |
| | | for nuclear reactor cooling |
| | Air Conditioning | Closed loop cooling for commercial and/or |
| | Commercialand | residential applications |
| | Residential | |
| | Supercomputer, | Closed loop cooling system for computer, |
| | computer, | supercomputer and other electronic, |
| | manufacturing, | manufacturing, processing, nuclear and other |
| | industrial, processing, | systems (e.g.—but not limited to—Bitcoin miners, |
| | etc. cooling | file managers, AWS, Microsoft, etc.) |
| Food | | |
| Related | | |
| | Livestock Processing | Temperature control within/below USDA |
| | | requirements |
| | Poultry Processing | Temperature control within/below USDA |
| | | requirements |
| | Fish Farm—post harvest | Temperature control within/below USDA |
| | processing | requirements |
| | Seafood Processing— | Temperature control within/below USDA |
| | maintainstemp | requirements |
| | throughout process line | |
| | Seafood Storage & | Temperature control within/below USDA |
| | Display | requirements |
| | Fruits & Vegetables in- | Temperature control within/below USDA |
| | field, transport, and | requirements |
| | process plant cooling | |
| | Fruit Freezing—Initial | In-field and transport cooling retains juices, |
| | Step to retain juices | prevents onset of bacterial growth and extends |
| | | shelflife |
| | Fruit Transportation— | Ideal temperature is reached prior to loading on |
| | Ground | truck. Temperature can be maintained throughout |
| | | ground transportation. Truck refrigeration not |
| | | used to cool down fruit - air cooling unnaturally |
| | | dries fruits and vegetables, but to maintain temp. |
| | | Moisture is retained. |
| | Beverage Cooling | Faster more efficient beverage cooling |
| | Bottles/Cans | |
| | Cold Storage—Proteins, | Temperature control within/below USDA |
| | fruits and vegetables | requirements |
| | SupermarketFresh | Temperature control within/below USDA |
| | Displays—fish, fruits, | requirements |
| | and vegetables | |
| | Restaurant Storage— | Temperature control within/below USDA |
| | seafood, fruits, veggies | requirements |
| | Bar Mixed Drink | Product can be manufactured with alcohol as the |
| | Application—alcohol | freezing depressant |
| | slurry | |
| | Bread & Cheese | Temperature control within/below USDA |
| | Processing | requirements |
| | Ice Cream Production | Ice cream contains water and salt, our slurry |
| | | solution may make smoother, creamier ice cream. |
| | Wine Production—Pectin/ | Immediate immersion after harvest is projected to |
| | tannin retention | increase tannin retention, strengthening aging |
| | | potential and possibly improving overall yields. |
| | Coffee Bean cooling | The faster beans are cooled after roasting the |
| | | more flavor they retain and the greater the flavor |
| | | profile when brewed. |
| Fire & Heat | | |
| Suppressant | | |
| | CommercialFire | Product density, BTU's, and pumpability make it |
| | Suppressant—buildings, | a candidate for building fire suppression systems |
| | homes, etc. | |
| | Forest Fire Suppressant— | Product density, BTU's, and pumpability make it |
| | air/ground | a candidate for fighting forest fires |
| | Metallurgy Quenching | Given that Product can be made with a variety of |
| | | fluids, potential exists for quenching |
| | 3-D Printer Quenching/ | Given that we can make Product with a variety of |
| | Cold Forge | fluids, potential exists for quenching |
| | Fire Fighter Pre- | Firefighter outerwear could be soaked in Product |
| | Treatment | for additional heat protection |
| Boating/ | | |
| Sea | | |
| Transport/ | | |
| Ocean | | |
| Freight | | |
| | Seawater Desalination | Potential applications |
| | Ocean OilSpill | Product can congeal certain oils and fuels |
| | Neutralization | |
| | 60 FT+ yacht Kitchen | Seafood preservation—up to 28 days depending |
| | Appliance | on species |
| | Cruise/FreightShip | Seafood preservation—up to 28 days depending |
| | Kitchen Appliance | on species |
| | Transport of Live | Extended chilling and preservation of live |
| | Shellfish, Fish, and other | lobsters and crustaceans, live finfish—sea life falls |
| | Seafood | asleep when placed in Product. Better and more |
| | | clean transportability. |
| | Preserved Transport of | Extended chilling and preservation of live |
| | Sea Life for Study | lobsters and crustaceans, live finfish - sea life falls |
| | | asleep when placed in Product. Better and more |
| | | clean transportability. |
| | Exhaust cooling to | 99% of boat engines are water cooled. This |
| | counteract water cooled | creates a large swath of hot water behind each and |
| | engines | every ship on the oceans. Combining this hot |
| | | water with Product could dramatically reduce |
| | | ocean warming. |
| Miscellaneous | | |
| | Weed Control | Weed control through Product application |
| | Horticulture | Product can be made with saline, and liquid |
| | Preservationand | fertilizers which allows for mixture with fertile |
| | Transportation | soil to keep roots and plant cool during transport. |
| | Ice Rink Repair | Fill holes and gashes on ice rink surface. |
| | Cement Mixing and | Significant amounts of cement (especially in |
| | Cooling | warmer climates) are wasted because it gets too |
| | | hot to bond when poured. Mixing Product and |
| | | other ingredients into the process water for |
| | | cement creation could aid in keeping the |
| | | temperature low enough for extended periods of |
| | | time |
| Government/ | | |
| CIA/ | | |
| Military/ | | |
| Police | | |
| | Crowd Control | Crowd dispersal through volume application. |
| | Enhanced Interrogation | Effective for discovery of critical information. |
| | Applications | |
| Cryo related | | |
| cellular | | |
| dissolution | | |
| | Cryolypolysis | Noninvasive fat reduction and toning. |
| | Cryoablation | Killing of cancerous tumors and other cellular |
| | | abnormalities when surgery not an option. |
| | | Cancers include, but are not limited to, bone, |
| | | cervical, eye, kidney, liver, lung, prostate and any |
| | | and any and all other forms. |
| Medical | Varicose Vein treatment | Product can reduce or eliminate varicose veins. |
| Other, | | |
| continued | | |
| (see above) | | |

### Additional Examples

As described herein, the cryogenic fluid composition that is frozen into the cryogenic fluid or slurry may include any alcohol, sugar, and/or salt. The alcohol, sugar, and/or salt have temperature lowering properties that allow for the nano-ice to form and maintains the cryogenic fluid as a slurry. Further, in one example, the salinity and percent weight to produce the nano-ice may be between 1.9% and 3.5% which produces freezing temperatures approximately between 20° Fahrenheit (F) and 31° F. (between −6.667 to −0.5 degrees Celsius).

In one example, the auger 800 may be positioned vertically so that frozen cryogenic fluid (e.g., nano-ice) may be elevated within the cryogenic fluid generator assembly 502 against the force of gravity resulting in the leaving of most of the unfrozen materials (e.g., water, cryogenic fluid compositions, etc.) behind and maintaining an ideal ice/water fraction in the frozen cryogenic fluid or slurry. Further, this ensures that unfrozen materials (e.g., water, cryogenic fluid compositions, etc.) may fall away as the nano-ice, frozen cryogenic fluid or slurry is lifted out of the dispensing spout 532 for deposition and application. At this ice/water fraction, the gel or slurry mixture may not be easily pumpable unless immediately mixed before dispensing and with dispensing chutes and hoses large enough not to clog.

In the examples described herein, the nano-ice, frozen cryogenic fluid or slurry may have a particle size with its above-mentioned inherent benefits at a size from approximately 200 nanometers (nm) to 500 nm in diameter. In one example, the particle size of the nano-ice, frozen cryogenic fluid or slurry may be between approximately 1 nm and 900 micrometers (μm). As a comparison, slurry ice seen in frozen uncarbonated beverages may be between 1 mm and 3 mm and does not have the size benefits of the nano-ice, frozen cryogenic fluid or slurry described herein. The surface of the nano-ice, frozen cryogenic fluid or slurry may include ridges, scratches, or cleavage points as initiation-sites crystal formation. The roughness criteria may be between at least 63 roughness average (RA) and 125 RA.

The diffusion ice crystal growth may be such that at least a crystal or wall of ice is formed to a thickness of at least 200,000 nm to 500,000 nm and the passing helical threads 804 and burrs 904 of the auger 800 may scrape off the ice crystals causing the ice crystals to break at the nanoscale. In comparison, if the auger gap were larger and the speed of rotation of the auger 800 were slower, the crystal formation may be larger, and ice may be formed on the 1 mm to 3 mm scale of slurry ice which does not hold the therapeutic benefits as described herein in connection with the nano-ice, frozen cryogenic fluid or slurry. The size of ice at the 200 nm to 500 nm (max 1 mm) may produce, for example, a replicate of a user's fingerprint.

The nano-ice, frozen cryogenic fluid or slurry being produced at this nanoscale also allows for dilation, reduction of swelling, and opening of pores of the user's skin to allow possible therapeutic agents in the nano-ice, frozen cryogenic fluid or slurry to pass into the skin topically. Similar effects may be experienced in connection with different types of tissues and organs. These therapeutic agents in the nano-ice, frozen cryogenic fluid or slurry may include, for example, methylsulfonylmethane (MSM), glucosamine, aloe including pure aloe, Epsom salts, trehalose, autologous cultured chondrocytes, cytokines for wound healing (e.g., derma gel, silvasorb, chlorhexidine 2%/4%, steroid creams), botulinum toxin type A, onabotulalinumtoxina (e.g., Botox), baclofen, tizanidine, cyclobenzaprine, iodine preparations (e.g., tincture of iodine, potassium iodide, iodophors), copper preparations (e.g., copper sulfate, copper naphthenate, cuprimyxin), sulfur preparations (e.g., monosulfiram, benzoyl disulfide), phenols (e.g., phenol, thymol), fatty acids and salts (e.g., propionates, undecylenates), organic acids (e.g., benzoic acid, salicylic acids), dyes (e.g., crystal [gentian] violet, carbolfuchsin), hydroxyquinolines (e.g., iodochlorhydroxyquin), nitrofurans (e.g., nitrofuroxine, nitrofurfurylmethyl ether), imidazoles (e.g., miconazole, tioconazole, clotrimazole, econazole, thiabendazole), polyene antibiotics (e.g., amphotericin B, nystatin, pimaricin, candicidin, hachimycin), allylamines (e.g., naftifine, terbinafine), thiocarbamates (e.g., tolnaftate), and miscellaneous agents (e.g., acrisorcin, haloprogin, ciclopirox, olamine, dichlorophen, hexetidine, chlorphenesin, triacetin, polynoxylin, amorolfine, Triclosan, Microban, Iodine, 0-phenylphenol, Hydronium, Dakin's Solution, hydrogen peroxide, honey, vinegar, essential oils, Erythromycin (e.g., antibiotics), mesenchymal stem cells (e.g., MSCs), platelet-rich plasma (PRP), autologous conditioned serum (ACS) and autologous protein solution (APS), chlorhexidine, dermatophilus congolensis, and combinations thereof, among other chemical compositions.

In one example, the cryogenic fluid composition may be formulated to allow for a number of formations including dendrites, plates, solid prisms, hollow prisms, solid columns, hollow columns, and needles, among other formations. In one example, the formations may be generated along the interior wall 1502 of the core 1206 at between approximately 0° to −5° C. range (approximately 32° F. to 23° F.). At this range of temperatures, the formations may be scraped or knocked off the interior wall 1502. In one example, the cryogenic fluid composition may be formulated as a supersaturation in grams per meter cubed (g/m³) at approximately 0 to 0.3 g/m³. The formation of the cryogenic fluid or slurry at these temperatures and supersaturation levels allows for the formulations described herein to form rather than, for example, relatively larger formulations. As the formations are scraped or knocked off the interior wall 1502, the formations may be subjected to shear forces that create even smaller formations such as the nano-ice formations described herein. Thus, in the first instance of creation, the formations may be relatively smaller, and the formations further decrease in size as they are scraped or knocked off the interior wall 1502.

### CONCLUSION

The examples described herein provide a systems, methods and formulations creating a therapeutic, homogeneous, cryogenic fluid. This simplified cryogenic system for cooling and freezing cryogenic fluid where the cryogenic fluid, once frozen, is scraped from an interior wall of a cylindrical housing by an auger housed and mechanically rotated within the cryogenic system is easy to operate and produces a superior therapeutic composition. The cryogenic system may include a heat exchange unit contained in an outer housing and in thermal coupling with the cylindrical housing and/or the auger. The present systems and methods further provide for a sealed, rapid heat exchange system including modular, self-aligning auger including a number of indexable end mills. Still further, the present systems and methods provide an angled push unit for expulsion of frozen material from the cylindrical housing of the cryogenic system. Further, the present systems and methods provide a number of chilling coils surrounding the auger and/or the cylindrical housing to freeze the cryogenic fluid in order to produce the therapeutic, frozen cryogenic fluid.

While the present systems and methods are described with respect to the specific examples, it is to be understood that the scope of the present systems and methods are not limited to these specific examples. Since other modifications and changes varied to fit particular operating requirements and environments will be apparent to those skilled in the art, the present systems and methods are not considered limited to the example chosen for purposes of disclosure and covers all changes and modifications which do not constitute departures from the true spirit and scope of the present systems and methods.

Although the application describes examples having specific structural features and/or methodological acts, it is to be understood that the claims are not necessarily limited to the specific features or acts described. Rather, the specific features and acts are merely illustrative of some examples that fall within the scope of the claims of the application.

What is claimed is:

1. A cryogenic fluid production device comprising:
a cylindrical housing;
a heat exchanger disposed within the cylindrical housing comprising:
an inlet;
a channel; and
an outlet,
wherein a coolant is conveyed through the inlet, the channel and the outlet of the heat exchanger; and
an interior wall; and
an auger disposed within the interior wall of the heat exchanger, the auger to remove material gathered on the interior wall.

2. The cryogenic fluid production device of claim 1, wherein:
the auger comprises at least one helical ridge that interfaces with the material gathered on the interior wall, and
the at least one helical ridge forces a cryogenic fluid composition introduced into an interior of the interior wall in a direction opposite a gravitational force.

3. The cryogenic fluid production device of claim 2, wherein:
a distance between the helical ridge of the auger and the interior wall is 0.005 in. to 0.015 in., and
the auger is rotated at a linear speed of between 15 mm/min and 3 mm/min.

4. The cryogenic fluid production device of claim 1, wherein the interior wall is textured.

5. The cryogenic fluid production device of claim 1, further comprising:
a processor; and
a non-transitory computer-readable media storing instructions that, when executed by the processor, causes the processor to perform operations comprising:
displaying, via a user interface, information defining a formulation of a cryogenic fluid introduced into the cryogenic fluid production device, a rotational speed of the auger, a status of a cryogenic fluid mixing process, a status of a cryogenic fluid cooling process, or combinations thereof.

6. The cryogenic fluid production device of claim 1, wherein the heat exchanger includes:
at least one spiral bar formed on a core, the at least one spiral bar forming at least one spiral chamber,
wherein:
the at least one spiral chamber forms the channel, and
the inlet and the outlet are fluidically coupled to the at least one spiral chamber.

7. The cryogenic fluid production device of claim 1, further comprising an ultraviolet germicidal irradiation (UVGI) assembly to sterilize at least one component of a cryogenic fluid composition.

8. The cryogenic fluid production device of claim 1, further comprising at least one filter to filter at least one component of a cryogenic fluid composition.

9. A therapeutic method, comprising:
generating a cryogenic fluid,
wherein:
the cryogenic fluid is formed between 0° to −5° C., and
the cryogenic fluid comprising at least one of:
water (H₂0); and
at least one salt,
wherein a ratio of water to the at least one salt is approximately between 1% and 6% salt with a remainder water; and
applying the cryogenic fluid to an organ tissue.

10. The therapeutic method of claim 9, comprising applying the cryogenic fluid directly to a tissue of an organ, indirectly to the organ tissue, or combinations thereof.

11. The therapeutic method of claim 9, wherein at least one of a temperature of the cryogenic fluid composition, a density of the cryogenic fluid composition, a viscosity of the cryogenic fluid composition, a size of solid particles within the cryogenic fluid composition or combinations thereof may be effected by adjusting at least one of a temperature of the cryogenic fluid composition as introduced into a cryogenic fluid composition device, a rotational speed of an auger within the cryogenic fluid composition device, a temperature of a heat exchange element of the cryogenic fluid composition device, or combinations thereof.

12. A cryogenic fluid composition, comprising:
water (H₂0); and
at least one salt,
wherein a ratio of water to the at least one salt is approximately between 1% and 6% salt with a remainder water.

13. The cryogenic fluid composition of claim 12, wherein the ratio is measured by weight or by volume.

14. The cryogenic fluid composition of claim 12, wherein a cryogenic fluid is formed from the cryogenic fluid composition at between 0° to −5° C.

15. The cryogenic fluid composition of claim 12, wherein the cryogenic fluid forms at least one of dendrites, plates, solid prisms, hollow prisms, solid columns, hollow columns, needles, or combinations thereof.

16. The cryogenic fluid composition of claim 12, wherein a shape of nanoparticles within the cryogenic fluid is:
at least one of approximately round, oblong, or globular; and
includes a roughness average (RA) of between 63 RA and 125 RA.

17. The cryogenic fluid composition of claim 12, wherein the diameter of nanoparticles within a cryogenic fluid formed from the cryogenic fluid composition is between 1 nanometer and 900 micrometers.

18. The cryogenic fluid composition of claim 12, wherein the at least one salt includes sodium chloride (NaCl) and magnesium sulfate (MgSO₄).

19. The cryogenic fluid composition of claim 12, further comprising at least one of an alcohol, a sugar, the at least one salt, or combinations thereof.

20. The cryogenic fluid composition of claim 12, further comprising at least one of methylsulfonylmethane (MSM), glucosamine, aloe including pure aloe, Epsom salts, trehalose, autologous cultured chondrocytes, cytokines for wound healing (e.g., derma gel, silvasorb, chlorhexidine 2%/4%, steroid creams), botulinum toxin type A, onabotulalinumtoxina (e.g., Botox), baclofen, tizanidine, cyclobenzaprine, iodine preparations (e.g., tincture of iodine, potassium iodide, iodophors), copper preparations (e.g., copper sulfate, copper naphthenate, cuprimyxin), sulfur preparations (e.g., monosulfiram, benzoyl disulfide), phenols (e.g., phenol, thymol), fatty acids and salts (e.g., propionates, undecylenates), organic acids (e.g., benzoic acid, salicylic acids), dyes (e.g., crystal [gentian] violet, carbolfuchsin), hydroxyquinolines (e.g., iodochlorhydroxyquin), nitrofurans (e.g., nitrofuroxine, nitrofurfurylmethyl ether), imidazoles (e.g., miconazole, tioconazole, clotrimazole, econazole, thiabendazole), polyene antibiotics (e.g., amphotericin B, nystatin, pimaricin, candicidin, hachimycin), allylamines (e.g., naftifine, terbinafine), thiocarbamates (e.g., tolnaftate), and miscellaneous agents (e.g., acrisorcin, haloprogin, ciclopirox, olamine, dichlorophen, hexetidine, chlorphenesin, triacetin, polynoxylin, amorolfine, Triclosan, Microban, Iodine, O-phenylphenol, Hydronium, Dakin's Solution, hydrogen peroxide, honey, vinegar, essential oils, Erythromycin (e.g., antibiotics), mesenchymal stem cells (e.g., MSCs), platelet-rich plasma (PRP), autologous conditioned serum (ACS) and autologous protein solution (APS), chlorhexidine, dermatophilus congolensis, or combinations thereof.
